(19) United States
(12) Patent Application Publication
(10) Pub. No.: US 2025/0025294 A1
(43) Pub. Date: Jan. 23, 2025

(54) A SUPPORTING STRUCTURE FOR A PROSTHETIC HEART VALVE, A RELATED HEART VALVE PROSTHESIS, AND A STORAGE KIT FOR A HEART VALVE PROSTHESIS

(71) Applicant: Corcym S.r.l., Milan (IT)

(72) Inventors: Felice Giuseppe CARLINO, Milan (IT); Marco BUSSONE, Milan (IT); Giovanni GIORDANO, Milan (IT); Roberto ZENZON, Milan (IT); Monica Francesca ACHILUZZI, Milan (IT)

(73) Assignee: Corcym S.r.l., Milan (IT)

(21) Appl. No.: 18/714,040

(22) PCT Filed: Nov. 24, 2022
(86) PCT No.: PCT/IB2022/061401

§ 371 Date: May 28, 2024

(30) Foreign Application Priority Data
Dec. 1, 2021 (WO) PCT/IB2021/061180

(51) Int. Cl. A61F 2/24

(52) U.S. Cl. CPC A61F 2/2418; A61F 2/2427; A61F 2220/0075

(57) ABSTRACT

Disclosed herein are improved supporting structures for heart valve prostheses that are collapsible to a low profile for delivery to the implantation site at the native heart valve. For example, the improved heart valve prostheses may be collapsed for minimally invasive delivery and then deployed/expanded at the native heart valve (e.g., mitral, aortic) for implantation. Advantageously, the collapsed configuration permits delivery through a small incision in the patient and also inhibits suture entrapment of the valve posts during implantation. The supporting structure includes an annular portion and a plurality of post members arranged along the annular portion that are collapsible for delivery and expandable for implantation. Also provided herein are delivery components that allow for delivery of the prosthesis.

### CROSS-REFERENCE TO RELATED APPLICATIONS

This application claims priority to PCT/IB2021/061180, filed Dec. 1, 2021, the entire contents of which are incorporated herein by reference.

### TECHNICAL FIELD

The present disclosure refers to implantable heart valve prostheses.

### BACKGROUND

Implantable biological valve prostheses are nowadays widely adopted in heart surgery thanks to undisputable advantages over mechanical heart valve prostheses. Over the years, this has resulted in a multitude of new surgical techniques for implantation of the prostheses.

The development of new surgical techniques progressed almost as rapidly as the—so to say—“shrinking” of the surgical access to the body of the patient. In other terms, practitioners and valve manufacturers have pushed towards minimally invasive approaches that no longer require a major surgical access such as a full sternotomy (open heart surgery), but rather require a very small access—often through the very vasculature—through which delivery facilities are routed.

As a result, while the implantation site has of course not changed, the operational area in the body of the patient has become more and more cramped and dark. While this can be countered through the design of the delivery facilities, there are still circumstances wherein accidents happen due to the features of the operational area. Implantation of biological prosthetic mitral heart valves is exemplary of such circumstances: biological mitral valve prostheses are typically delivered in a minimally invasive manner through a delivery technique commonly known as “parachuting”, wherein a plurality of threads are preliminarily routed at and through the implantation site (a valve annulus) to provide both a travel guide during approach to the implantation side by the valve, and suturing locations. Parachuting threads go through the annulus and loop back through the sewing cuff of the prosthesis to provide guidance. Parachuting threads usually loop distally of the annulus in the implantation direction, i.e. at an area which corresponds to the final intended location of the valve posts once implanted. This per se is already susceptible of causing accidental capturing of a valve post by a loop—even before the same is tightened into a suture, but such a risk is considered inherent in the implantation technique and can be kept reasonably under control. However, in real life conditions practitioners tend to encourage expansion of the implantation site to implant a prosthesis as big as possible (leaving the valve leaflets in place means that the flow section is inherently lower than the native one) to decrease pressure drops across the prosthesis itself, and the only end effector available at that location is the prosthesis mounted onto the delivery instrument. The prosthesis is manipulated at the annulus with an essentially wobbling motion around the axis of the annulus in an effort to expand the latter, which results in a wobbling motion of the valve posts accordingly. Under these circumstances, even when best efforts are made to keep the loops of the parachuting threads away from the valve posts, the latter move relative to the parachuting thread loops to the point that they usually become entangled in the thread loop(s). When the threads are tightened into a suture, the entangled posts are no longer visible by the practitioner as they are located on an opposite side of the annulus from that of the surgical access, whereby vision is obstructed by the prosthesis itself. As a result, tightening of the thread into a suture entraps the valve post into the loop it was already entangled by, and the newly implanted valve prosthesis is not in a condition to provide full coaptation of the prosthetic valve leaflets. The prosthesis therefore suffers from regurgitation literally moments after it is implanted, and either requires immediate explantation or—when suture entrapment is not severe—will have an expected service life approximately ten times shorter than design specifications.

Some attempts have been made in the art to at least partially control the position of the valve posts during implantation, but these attempts are largely unsuccessful: the valve posts cannot be deflected or otherwise manipulated to a sufficient extent to keep them off the parachuting suture loops, whereby suture entrapment occurs regardless of all the efforts.

U.S. Pat. No. 5,554,185 to Block describes an inflatable prosthetic cardiovascular valve which is constructed so as to be initially deployable in a deflated “collapsed” configuration wherein the valve may be passed through the lumen of a cardiovascular catheter and subsequently inflated to an “operative” configuration so as to perform its intended valving function at its intended site of implantation within the cardiovascular system.

### SUMMARY

Provided herein are improved supporting structures for heart valve prostheses that are collapsible to a low profile for delivery to the implantation site at the native heart valve. For example, the improved heart valve prostheses may be collapsed for minimally invasive delivery (e.g., through a small incision in the patient, between two ribs of the patient, and to the heart) and then expanded at the native heart valve for implantation. Advantageously, the collapsed configuration inhibits suture entrapment of the valve posts in the implantation of biological or biological-like heart valve prostheses, for example mitral heart valve prostheses. Additionally, the improved support structures and heart valve prostheses herein may facilitate implantation even with procedures wherein suture entrapment is inherently mitigated, such as aortic valve prostheses. In such procedures, the improved heart valve prostheses herein may facilitate attachment to the native annulus by way of devices involving the use of a single loop wire snare to pull suture ends through a mechanical knot.

Also provided herein are delivery components that allow for delivery of the prosthesis through a narrower surgical access as compared to prior art solutions, while also inhibiting suture entrapment.

An improved supporting structure for the prosthetic heart valve permits the heart valve prostheses to be collapsible to the low profile for delivery. The prosthetic heart valve also includes prosthetic leaflets coupled to the supporting structure. Further, a storage kit for a heart valve prosthesis is provided. Details on the features may be found in the claims that follow, which form an integral part of the technical disclosure provided herein.

### BRIEF DESCRIPTION OF THE FIGURES

Further features and advantages of the disclosure will become apparent from the following description with reference to the annexed figures, provided purely by way of non-limiting example, wherein:

FIG. 1 is a perspective view of a supporting structure according to embodiments of the disclosure, in a first operating condition,

FIGS. 2, 3, 4, and 5 are views according, respectively, to pointers II, III, IV, and V in FIG. 1,

FIG. 6 is a perspective view of a supporting structure according to embodiments of the disclosure, in a second operating condition,

FIG. 7 is a view according to pointers VII in FIG. 6,

FIGS. 8A to 8F show further available configurations of the supporting structure embodiments herein,

FIGS. 9A and 9B are views according, respectively, to pointers VIII and IX in FIG. 6,

FIGS. 10 and 11 are sectional views according to lines X-X and XI-XI in FIGS. 1 and 7, respectively,

FIGS. 12, 13, 14, and 15 are views of a heart valve prosthesis comprising a supporting structure according to embodiments of the disclosure, respectively under the angles of view of FIGS. 4, 5, 9A, and 9B, and in the same conditions accordingly,

FIGS. 16A-16C and 17A-17C are perspective views of further embodiments of a supporting structure according to the disclosure,

FIGS. 18 and 19 are perspective views of yet further embodiments of the supporting structure according to the disclosure,

FIGS. 20A-20C and 21A-21C are perspective views of yet further embodiments of the supporting structure according to the disclosure, in different conditions,

FIGS. 22A-22C and 23A-23C are perspective views of the embodiments of FIGS. 20A-20C and 21A-21C with a heart valve prosthesis mounted on the supporting structure, with FIG. 23D featuring a detail of FIG. 23C,

FIG. 24 is a plan view of an arrangement of components of the embodiments of FIGS. 20 to 23,

FIG. 25 is an exploded perspective view including the components of FIG. 24,

FIGS. 26 to 28 show sectional views corresponding to an operating sequence involving the components and features of FIGS. 20 to 25, and

FIGS. 29 to 33, 34 to 37, and 38 to 42 show respective embodiments of a system configured for use with the prostheses and the supporting structures disclosed herein.

### DETAILED DESCRIPTION

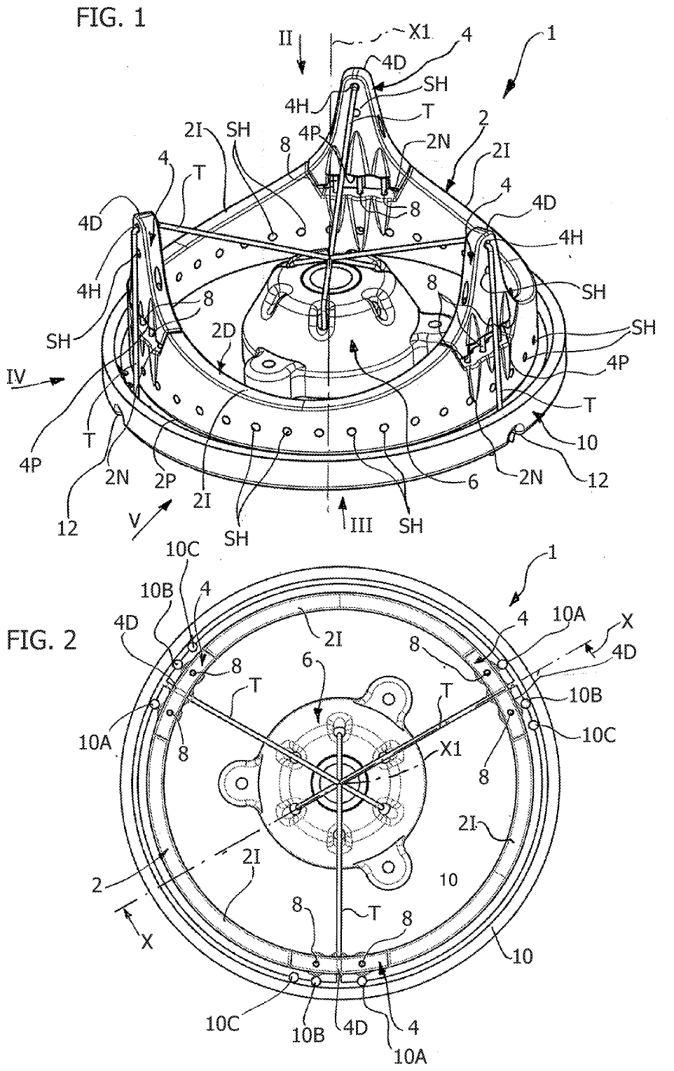

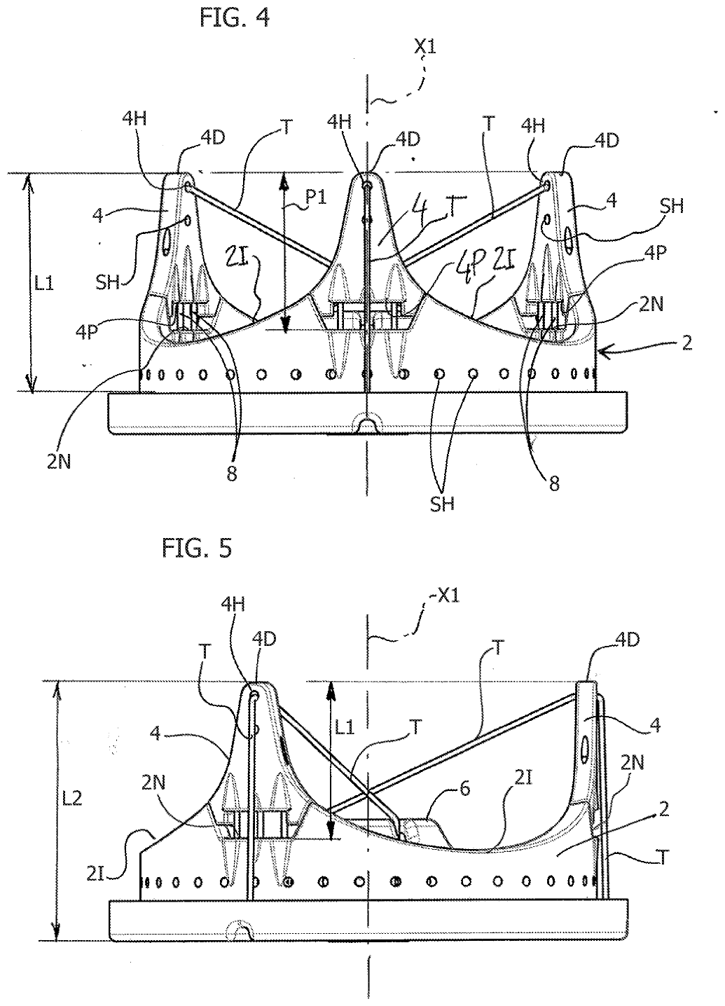

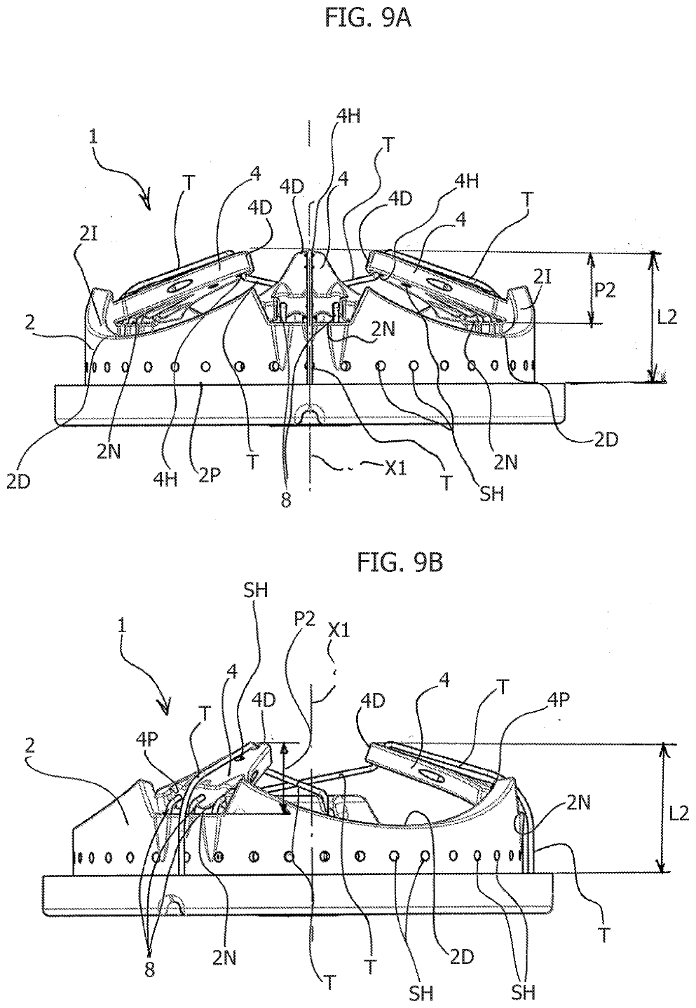

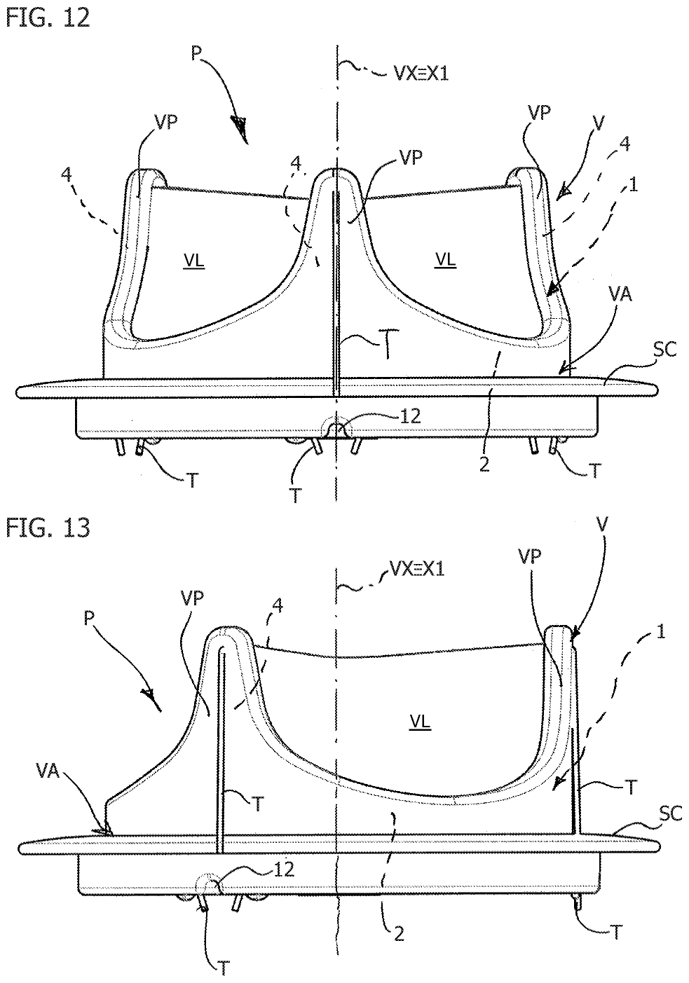

Reference number 1 in FIGS. 1 through 15 designates as a whole a supporting structure according to embodiments for a prosthetic heart valve V (FIGS. 12 to 15) comprising a prosthetic valve annulus VA defining a valve longitudinal axis VX and a plurality of prosthetic valve leaflets VL extending between subsequent valve posts VP arranged along the prosthetic annulus VA and around the valve axis VX.

In embodiments, the prosthetic heart valve V may be a three-leaflet valve made of biological tissue such as bovine pericardium, or a polymer fabric, or may even feature a hybrid construction wherein the leaflets VL are made of biological tissue and the remainder of the prosthesis is made of a polymer fabric. As a general remark, “made of” is to be generally understood as comprising the material concerned, i.e. leaving room for other materials intervening in the construction of valves such as the valve V (for instance suture threads or the like).

In embodiments, the valve V may be a fully biological prosthetic heart valve made of biological tissue such as bovine pericardium. In other embodiments, the valve V may have a construction identical to the biological embodiments (biological-like), but is made of a polymer fabric, or a combination of a polymer fabric for the leaflets VL and an elastic or stretchable fabric (either polymer based or not) for the remainder of the valve V.

In yet other embodiments, the valve V may feature a hybrid construction wherein, for instance, the valve leaflets VL are made of biological tissue and the remainder of the valve is made of a polymer fabric or an elastic or stretchable fabric (either polymer based or not).

As to biological tissues, those most commonly used for the manufacturing of the valve V may comprise a decellularized sterilized mammalian tissue having an extracellular matrix, either of the “wet” type, or of the “dry” type, i.e. wherein a plurality of interstitial spaces of the extracellular matrix include a solution of one or more polyols, typically glycerol.

As said, such biological tissues are generally used for manufacturing the leaflets VL, while the remainder of the prosthesis may comprise the biological tissue itself or a polymer fabric, or else an elastic or stretchable fabric

In one embodiment, both the valve leaflets are made of a decellularized sterilized mammalian tissue (e.g. bovine pericardium) having an extracellular matrix, wherein a plurality of interstitial spaces of the extracellular matrix include a solution of one or more polyols, typically glycerol. This results in a so-called “dry” valve V, i.e. a valve V allowing dry storage thereof without a liquid preservation solution as it is the case with “wet” valves made out of or including portions of “wet” biological tissue.

With reference to FIGS. 1 to 11, in embodiments the supporting structure 1 includes:
an annular portion 2 defining a supporting structure longitudinal axis X1 (in other terms, the annular portion 2 includes a longitudinal axis X1 that defines the supporting structure longitudinal axis, i.e. the longitudinal axis of the supporting structure 1), and
a plurality of post members 4 arranged along the annular portion 2 around the supporting structure longitudinal axis X1, wherein each post member 4 extends from a proximal end 4P to a distal end 4D, the distal 4D end having a protrusion with respect to the annular portion in a direction parallel to the axis X1. Each post member 4 is movable with respect to the annular portion 2 so as to vary the protrusion of the distal end 4D thereof from a first protrusion P1 to a second protrusion P2, lower than the first protrusion P1.

The supporting structure also may include a drive member 6 operatively connected to the post members 4 and operable to move the post members 4 between the first protrusion P1 and the second protrusion P2.

As a general remark, indicating that embodiments herein may feature a drive member operable to move the post members 4 between the first protrusion P1 and the second protrusion P2 includes the following range of possibilities.
i) From P1 to P2 (for instance, and in no way as a limitation, embodiments 1, 100)
ii) From P2 to P1 (for instance, and in no way as a limitation, embodiments 300)
iii) From P1 to P2 and vice versa (for instance, and in no way as a limitation, embodiments 200).

In embodiments, the annular portion 2 may be configured to fit at the prosthetic valve annulus VA, and each of the post members 4 may be configured to fit at a corresponding valve post VP of the heart valve prosthesis V.

In embodiments, the supporting structure 1 may include three post members 4, and the prosthetic valve V may include three valve posts VP (and three valve leaflets VL) accordingly.

As regards the protrusions P1, P2, in embodiments the same result in an overall axial length of the prosthesis measured along the axis X1 which varies consistently with the protrusion, whereby the protrusion P1 is associated to a first axial length L1, and the protrusion P2 is associated to a second axial length L2, the axial length being measured between the distal end(s) 4D and a proximal end (or rim) 2P of the annular portion, while a distal end (or rim) 2D of the annular portion 2 faces or is adjacent to the proximal ends 4P of the post member(s) 4. As to the protrusions P1, P2, they may be defined differently based on the embodiments herein, and in general they may be regarded as an indication of an axial extension of the post members 4 off the annular portion 2. In general terms, the condition corresponding to the protrusion P1 may be regarded as a “deployed” condition of the post members 4, while the condition corresponding to the protrusion P2 may be regarded as a “collapsed” condition of the post members 4. Reference to the “deployed” and the “collapsed” condition may be made throughout this disclosure in place of reference to protrusions P1 and P2, with the respective equivalent meaning.

Advantageously, the supporting structure described herein is transitionable from the collapsed configuration for delivery to the deployed configuration for implantation without the need for any inflation. Thus, unlike the Brock patent described above that requires inflation, the prostheses described herein can expand without inflation tools, thereby avoiding additional tools and structures that increase the size of the implant and make it more difficult for minimally invasive delivery. In some embodiments, each of the post members (4; 4A; 104; 204; 304) is configured to move with respect to the annular portion (2; 2A; 2B; 102; 202; 302) from the second protrusion (P2) in the collapsed configuration for delivery to the first protrusion (P1) in the deployed configuration for implantation without any inflation.

In the embodiments of FIGS. 1 through 15, each post member 4 is cantilevered to the annular portion 2 at the proximal 4P end thereof, i.e. a cantilever connection is provided between the proximal end 4P and the distal end 2D of the annular portion 2.

In embodiments the cantilever connection may include one or more sections 8 of a wire-like member. In embodiments, three sections 8 of a wire like member extend in a bridge like fashion between the end 4P and the end 2D. In embodiments, the wire like member of each section 8 is made of a superelastic material.

In these embodiments, the annular portion 2 may be shaped as a ring having a substantially flat rim at the proximal end 2P and a multi-concave rim at the distal end 2D, which has accordingly an axial location that is variable around the axis X1. Particularly, the distal end 2D may include (all parts of the end 2D) a combination of inter-post portions 2I having a concave or scalloped shape, alternated with notch portions 2N at the post members 4. In the embodiments herein, purely by way of example, the annular portion 2 comprises three notch portions 2N and three inter-post portions 2I.

In these embodiments, the protrusions P1, P2 may be defined based on the notch portions 2N, as the same are generally provided with a flat bottom making it easier to provide a reference for measuring a distance. Protrusions P1 and P2 may therefore be defined as the distance along the axis X1 from the distal end 2D at the bottom of the notch portion 2N and the distal end 4D of the post member 4 whose proximal end 4 faces the notch bottom.

Of course, the definition may be formulated based on other points of the distal end 2P, even in the scalloped inter-post portions 2I, but—again—the notch 2N is preferred for simplicity. In general terms, it may not be the actual value of the protrusions P1, P2 that matters, but rather the provision of post members 4 with a variable offset with respect to the annular portion 2, and an overall supporting structure 1 with a variable axial length. With reference again to FIGS. 1 through 15, in embodiments the supporting structure 1 may further comprise a holder 10 releasably coupled to the annular portion 2. The holder 10 is arranged at the proximal end 2P of the annular portion 2, may be coupled thereto at the same location.

In embodiments, the holder 10 extends across the annular portion 2, and in some embodiments it may actually span the entire area of the lumen defined by the annular portion 2 at the proximal end 2P thereof, thereby closing the lumen. In such embodiments, the holder 2 may comprise a circular plate coaxial to the axis X1.

In embodiments wherein the holder does not span the entire area of the lumen of the annular portion 2, the holder 10 may comprise a hub at the axis X1 (and coaxial thereto) and a plurality of spokes—for instance having the same number as the post members 4 reaching the annular portion 2 from the hub.

In embodiments—regardless of the features of the holder 10 as disclosed above—the drive member 6 may be arranged on the holder 10, specifically at the axis X1 (at the hub in case the holder 10 is configured with the hub and spoke layout as above).

In such embodiments, the drive member 6 is connected to each support post by means of a respective thread T. Each thread has a first end coupled to the drive member 6, and is coupled to the respective post member 4 at the distal end 4D thereof. As visible in FIGS. 1 through 15, coupling of the thread T to the distal end 4D may occur by routing the thread T through a distal through hole 4H at the distal end 4D.

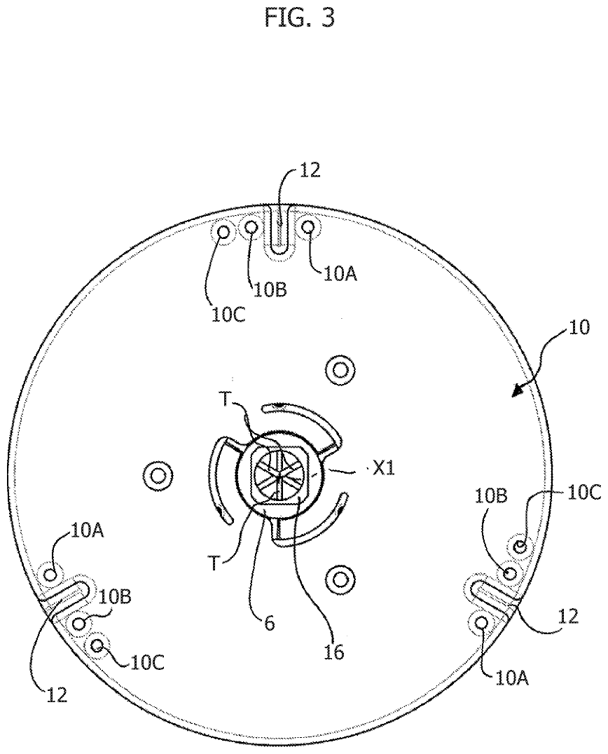

In embodiments, the second end of each thread T is coupled to the holder 10. Specifically, each thread T goes through the holder 10 to expose the second end at an opposite side of the holder 10 from the supporting structure 1. As visible for instance in FIG. 3, the holder 10 may include—at locations corresponding to those of the post members 4 of the supporting structure 1, pattern of through holes 10A, 10B, 10C configured to receive the thread T at the holder 10. In one embodiment, each thread T runs from the first end at the drive member 6, extends radially—or away from the axis X1—to (and through) the post member 4, then goes axially to the holder 10 along the outer surface of the post member 4 (note, however, that the thread may optionally go through the valve posts VP as well, as shown in FIGS. 12 to 15, therefore running on the outer surface of the valve posts), the annular portion 2 and through the hole 10A to exit on an opposite side of the holder 10 with respect to the annular portion 2 (and the posts 4). From here, the thread T may loop back into the hole 10B and exit on the side of the annular portion 2, only to loop back again in through the hole 10C at the second end thereof. The thread T may then be tied to secure the holder 10 to the supporting structure 1.

In such embodiments, the thread T exposes a cutoff section between holes 10A and 10B on the side of the holder 10 visible in FIG. 3, i.e. on an opposite side of the holder 10 than the annular portion 2. This renders the cutoff section accessible even after positioning the prosthesis, as the same is generally delivered in a proximal to distal direction, whereby the side of the holder opposite the supporting structure 1 (and the prosthesis V overall) remains oriented proximally during implantation.

In embodiments, radial notches 12 may be provided between each pair of holes 10A-10B to accommodate a cutting instrument to severe the thread at the cutoff section.

In embodiments, the second ends of each thread may be generally loop-tied to the holder 10 to expose a cutoff section, wherein severing or cutting of each thread T at the cutoff section releases the coupling between the holder 10 and the supporting structure 1, and also leaves the post members 4 in a position having the first protrusion P1, i.e. in the deployed position.

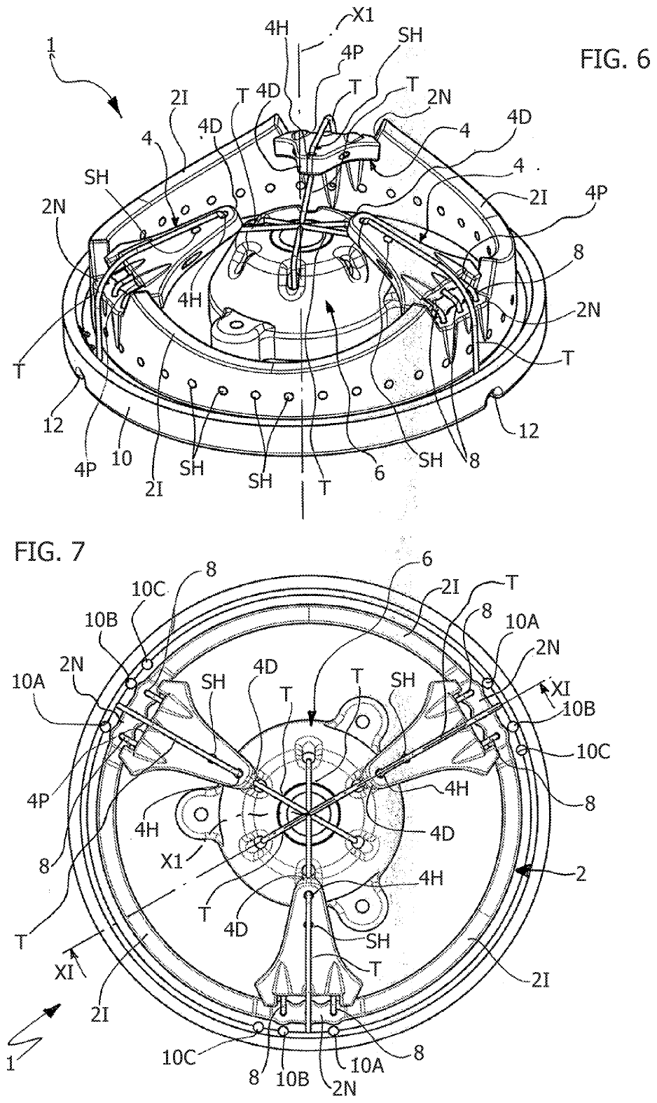

In embodiments, the drive member 6 is configured to attract the threads T towards the supporting structure longitudinal axis X1 to orient the distal ends of the post members 4 thereof towards the axis X1, thereby transitioning from the first protrusion P1 (FIGS. 1, 2, 4, 5, 8A-8F, 10, 12, 13) to the second protrusion P2 (FIGS. 6, 7, 9A, 9B, 11, 14, 15).

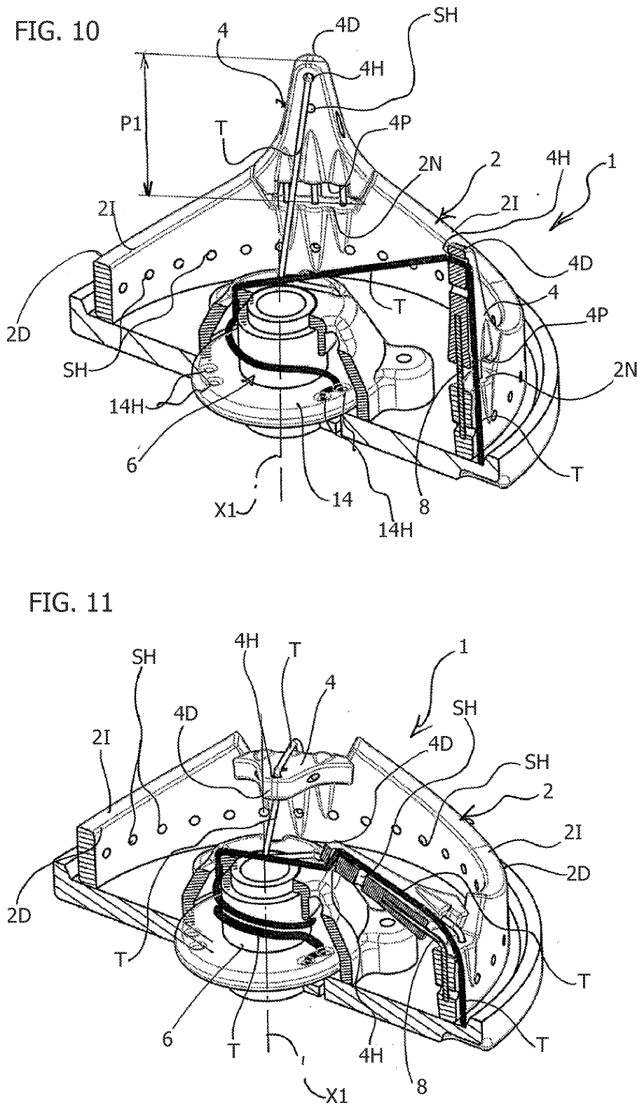

In embodiments, the drive member 6 may comprise a rotary ratchet drive member including a drive hub 14 (ratchet hub) coaxial to the axis X1, wherein the first end of each thread is coupled to the rotary ratchet drive member and particularly to the hub 14 via through holes 14H, single or paired. In embodiments, when the post members are arranged so as to have the first protrusion P1, the threads T are arranged so that the first end thereof is attached to the hub 14 on an opposite side of the axis X1 than the post member 4 the threads is coupled to (FIG. 10).

In embodiments the drive hub 14 is configured to be engaged by a drive instrument for operation of the drive member 6. Engagement may occur for instance via a drive socket 16 (FIG. 6), which may be arranged so as to face away from the supporting structure 1 in a proximal direction in order to be accessible during implantation, i.e. to be accessible from an opposite side of the holder 10 than the supporting structure 2.

With reference to FIGS. 12 through 15, a heart valve prosthesis P in embodiments comprises the prosthetic valve V mounted onto the supporting structure 1 so that each post member 4 fits at a corresponding valve post VP and the annular portion 2 generally fits at the annulus VA. A sewing cuff SC may be featured on the valve V that extends at the annulus VA or slightly proximal thereof.

Specifically, the supporting structure 1 may be regarded functionally as a valve stent for the prosthetic heart valve V and may be arranged within the lumen of the valve V, whereby the post members 4 are simply sutured to the inner surface of the valve posts VP and the inner surface of the valve annulus VA, or may be configured as a core of the valve V, whereby the entire support structure is enclosed or “wrapped” by the valvular sleeve of the valve V, except for the leaflets VL.

Suturing of the valve V to the supporting structure 1 may be provided thanks to sewing holes SH provided both on the post members 4 and on the annular portion 2 (in a circumferential arrangement around the axis X1), whereby a stable connection between the valve V and the supporting structure 1 is achieved. In embodiments, suturing may occur at the posts 4 only, with the remainder of the valvular sleeve of the valve V left unconstrained and wrapping the supporting structure 1.

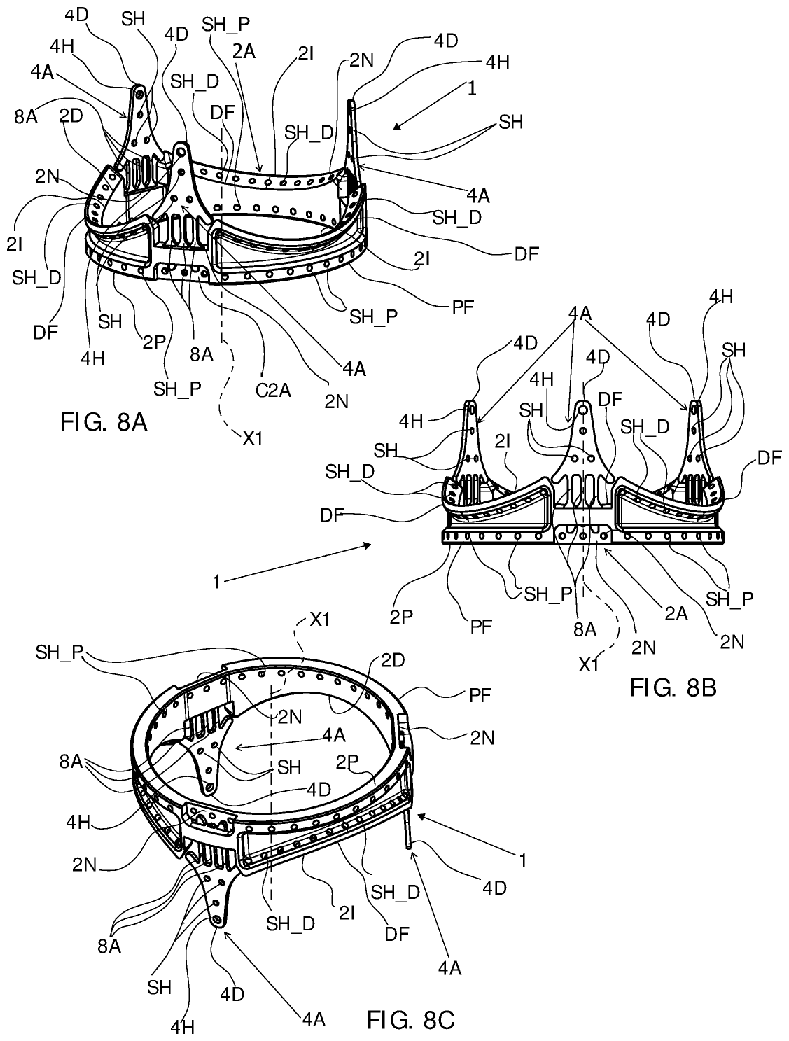

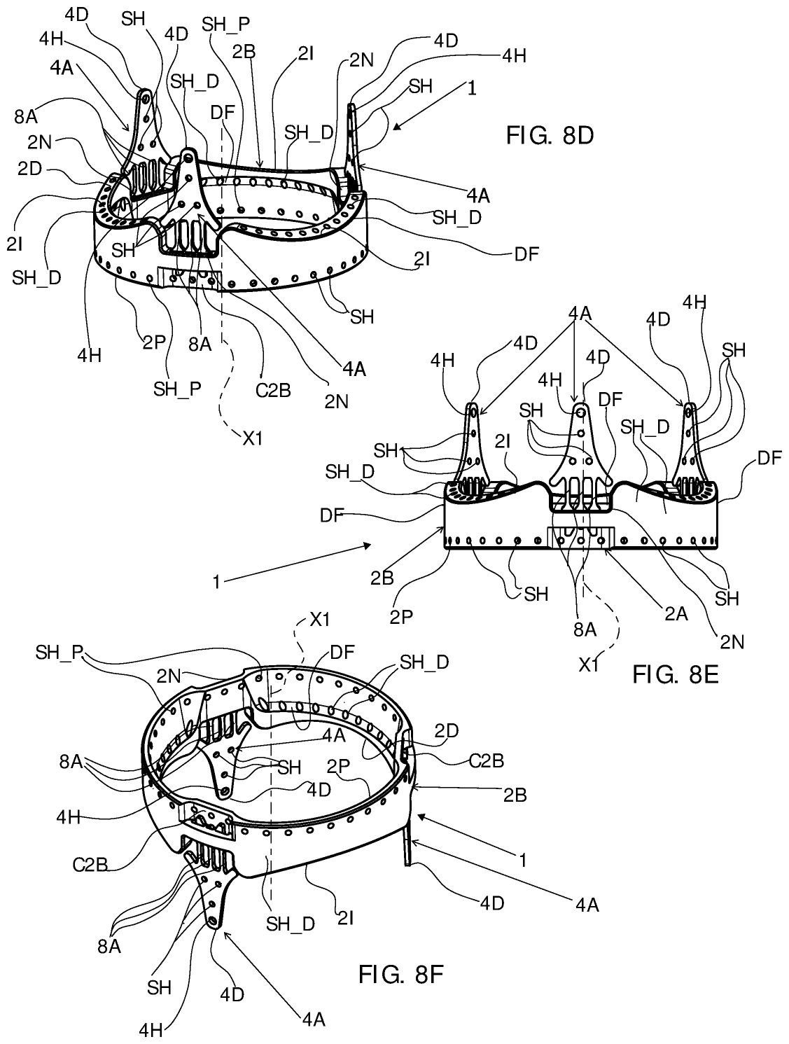

With reference to FIGS. 8A to 8F, wherein reference numbers identical to those used in the foregoing designate the same components, the cantilevered connection of the post members to the annular portion 2 may be provided by post members 4A integrally formed with extensions 8A arranged similarly or identically to the sections 8 disclosed in the foregoing and visible in FIGS. 1 through 7. Thus, extensions 8A also may be one or more sections of wire-like members so that each of the post members is cantilevered to the annular portion through the one or more sections of a wire-like member. In these embodiments, the post members 4A retain the swept-back shape of the post members 4, as well as the location of holes SH and 4H, but are formed in one piece of the same superelastic material (e.g. Nitinol) as the extension 8A, whereby each post member features one or more extensions 8A at the proximal end 4P thereof. The one or more extensions 8A are fitted into a corresponding seat in the annular portion 2, particularly at the notch portions 2N, so to result in a cantilevered connection wherein the distal end 4D of the post 4A is movable with respect to the annular portion to define the protrusions P1, P2 via the flexing of the one or more sections 8A.

The post members 4A may be provided in combination with the annular portion 2 with the features disclosed in the foregoing—and visible in FIGS. 1 through 7—or in combination with an annular portion 2A (FIGS. 8A to 8C; the resulting supporting structure is referred to as 1A), or else in combination with an annular portion 2B (FIGS. 8D to 8F; the resulting supporting structure is referred to as 1A), the features of both of which will be detailed in the following. Similarly, in embodiments the post members 4 and sections 8 may be provided in combination with the annular portions 2A and 2B.

The annular portion 2A retains the basic structures and features of the annular portion 2 such as the notch portions 2N, the inter-post portions 2I, and the proximal and distal ends 2P, 2I. The annular portion 2A differs from the annular portion 2 in that additionally features a proximal flange PF at the proximal end 2P and a distal flange DF at the distal end 2D. In embodiments, the proximal and distal flanges PF, DF protrude outwardly of the annular portion 2A, and are essentially provided as three consecutive flange section only interrupted by the notch portions 2N. In embodiments, the proximal flange PF extends transversely outwardly of the annular portion 2A at the proximal end 2P, while the distal flange DF extends outwardly of the annular portion 2A at the distal end 2D with a flared (as per the figures) or a tapering pattern (the latter not visible in the figures, but essentially a reversed flared pattern, with the sections of the flange DF swept back towards the proximal end 2P).

Each of the proximal and distal flanges PF and DF includes a respective pattern of sewing holes, namely a pattern of proximal sewing holes SH_P on the proximal flange PF and a pattern of distal sewing holes SH_D on the distal flange DF. All of the sewing holes SH_P and SH_D are through holes extending through the respective flange PF, DF, wherein the holes SH_D extend through a thickness (axial thickness) of the flange DF, while the holes SH_P extend radially through the flange PF.

The proximal sewing holes SH_P have a similar arrangement to the sewing holes SH, as they are arranged along a circumference centered on the axis X1 and with respective axes oriented along a direction radial to the axis X1 (i.e. transverse to the axis X1). In embodiments, however, it may be envisaged to provide the holes SH_P with axes slightly angled with respect to a pure radial direction. The proximal sewing holes SH_P are also provided at cutaways C2A located at the notch portions 2N below the seat where the one or more extensions 8A are fitted.

The distal sewing holes SH_D are arranged according to a circumferential pattern as well, however they are only provided on the distal flange DF, whereby the pattern is interrupted at the notch portions 2N. the axes of the sewing holes SH_D are generally oriented so as to be orthogonal to the (distal) surface of the flange DF, but slight offset from an orthogonal condition may be envisaged.

Similarly to the holes SH_P, the holes SH_D are through holes intended to receive suture threads as coupling members connecting the valve V to the supporting structure 1 (when the post members are embodied as the post members 4), 1A. in detail, the proximal sewing holes SH_D are configured for the routing of suture threads therethrough to secure a wrapping cloth or sheath (e.g. made of fabric) around the proximal end of the annular portion, for instance the sewing cuff SC, while the distal sewing holes SH_D are configured for the routing of suture threads securing together the leaflets VL and a wrapping cloth or sheath (e.g. made of fabric) that covers the post members 4. Each of the flanges PF, DF offers a secure, stable attachment for the suture threads which facilitates manufacturing of the prosthesis P, and the distal flange DF also provides a more robust attachment interface for the leaflets VL which also helps in reducing leaflet stress when the post members 4A are operated to reach the second protrusion P2, and more in general when the condition of the post members is changed to transition from one protrusion to another.

The annular portion 2B also retains the basic structures and features of the annular portion 2 such as the notch portions 2N, the inter-post portions 2I, and the proximal and distal ends 2P, 2I. The annular portion 2B differs from the annular portion 2, as well as the annular portion 2A, in that it features a proximal flange PF at the proximal end 2P, and no distal flange DF. Additionally, unlike the annular portion 2B, the proximal flange protrudes inwardly of the annular portion 2B. In embodiments, as seen for instance in FIG. 8F, the inward protrusion is provided by tapering the inner wall of the annular portion 2B in a proximal-to-distal direction while keeping the outer wall parallel or essentially parallel to the axis X1, which corresponds to increasing the wall thickness of the annular portion 2B in a proximal-to-distal direction at the sections including the distal flange DF. This i.a. avoids taking away too much of the cross section for blood (in)flow through the prosthesis P just to make the flange DF, while also retaining a radial stiffness on the annular portion 2B.

Similarly to the distal flange DF of the annular portion 2A, the distal flange DF of the annular portion 2B is interrupted at the notch portions 2N, each featuring a cutaway C2B on the outer surface of the annular portion 2B. The cutaways C2B also result in a mild inward protrusion of the notch portions 2N altogether, which generates the interruption of the flange DF. A circumferentially continuous flange DF may be nevertheless envisaged for the annular portion 2B.

In embodiments, the distal end 2D of the annular portion 2B is essentially orthogonal to the axis X1, but other orientations may be envisaged, i.e. flared or tapering.

The proximal distal flange DF includes a respective pattern of distal sewing holes SH_D, while the sewing holes already disclosed in respect of the annular portion 2 are arranged at or near the proximal end 2P and represent—accordingly—proximal sewing holes.

The distal sewing holes SH_D on the distal flange are through holes extending through the thickness of the flange DF and have axes parallel to the axis X1 or lying at an angle offset from zero angle (i.e. offset from a parallel condition), for instance 30 to 45 degrees (endpoints included, and end points being representative configurations) with respect to the axis X1. In general, the open ends of the (through) sewing holes SH_D are such that they are completely contained within the distal flange DF, i.e.—considering a distal-to-proximal direction—they lead in at the distal end 2D and lead out at the undercut created by the inwardly protruding distal flange DF.

The distal sewing holes SH_D are arranged according to a circumferential pattern, similarly as the holes SH, SH_P, however they are only provided on the distal flange DF, whereby the pattern is interrupted at the notch portions 2N.

Similarly to the holes SH, the holes SH_D are through holes intended to receive suture threads as coupling members connecting the valve V to the supporting structure 1 (when the post members are embodied as the post members 4), 1B. In detail, the proximal sewing holes SH are configured for the routing of suture threads therethrough to secure a wrapping cloth or sheath (e.g. made of fabric) around the proximal end of the annular portion, for instance the sewing cuff SC, while the distal sewing holes SH_D are configured for the routing of suture threads securing together the leaflets VL and a wrapping cloth or sheath (e.g. made of fabric) that covers the post members 4. The flange DF offers a secure, stable attachment for the suture threads which facilitates manufacturing of the prosthesis P, and provides a more robust attachment interface for the leaflets VL which also helps in reducing leaflet stress when the post members 4A are operated to reach the second protrusion P2, and more in general when the condition of the post members is changed to transition from one protrusion to another. Operation of the embodiments disclosed in the foregoing will now be disclosed.

In embodiments, the heart valve prosthesis comprising the prosthetic valve V mounted onto the support structure 1, the latter including the holder 10, is provided as a pre-assembled item to the practitioner.

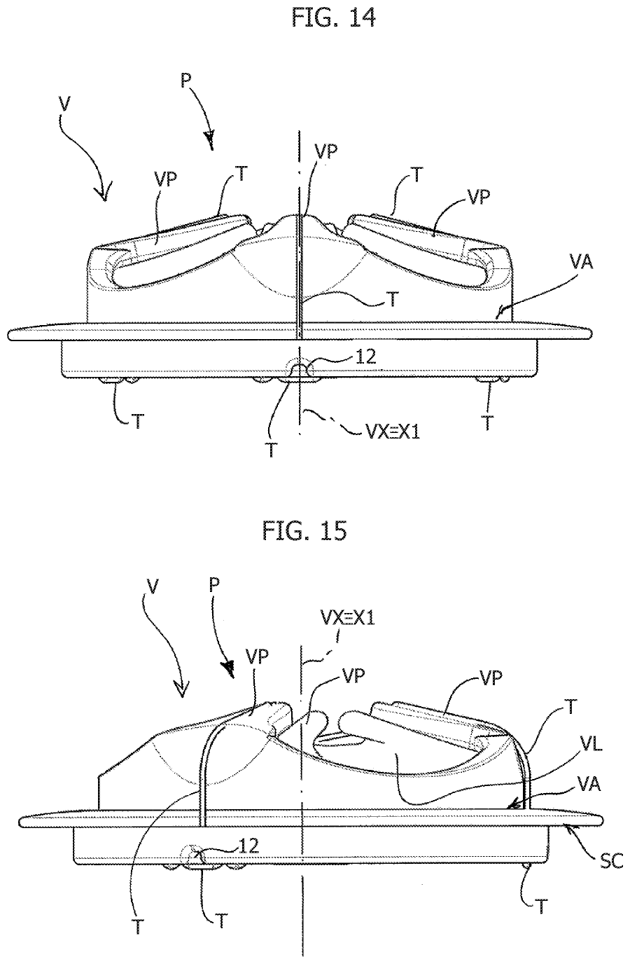

The heart valve prosthesis P may be provided with the supporting structure 2 configured as shown in FIGS. 12, 13, i.e. with the post members 4 n the deployed position (featuring the first protrusion P1=, but particular advantages may be achieved when the in the initial condition of the heart valve prosthesis the supporting structure 1 is configured as visible in FIGS. 14, 15, i.e. so that the posts members 4 are in the collapsed position (featuring the second protrusion P2).

In the latter condition, the valve posts VP are essentially folded radially within the lumen of the valve V (and so are the post members 4 relative to the annular portion 2), whereby the prosthesis has an overall axial length which is much shorter than the axial length it would have, were it arranged as shown in FIGS. 12, 13.

In embodiments, the heart valve prosthesis P comprising the prosthetic valve V mounted onto the support structure 1 is provided in an initial condition wherein the post member 4 are arranged so to have the second protrusion P2, i.e. in the collapsed position with the valve posts VP folded radially within the lumen of the valve V. In embodiments the holder 10 is provided, too, as it may carry the drive member 16, and receive the threads T.

This condition may be pre-set at the manufacturing stage after mounting the valve V onto the supporting structure 2 (which occurs with the supporting structure in a non-stressed condition, i.e. with the post members in the deployed position), or may be achieved directly in the operational field by engaging the drive socket 16 with a drive instrument. This rotates the drive hub 14 and winds the threads T onto the hub itself, thereby tensioning and attracting the thread towards the axis X1. Tensioning of the threads T occurs owing to the resistance to bending (or generally resistance to folding) from the post members 4 as winding of the threads onto the hub 16 progresses.

As the threads T are engaged with the post members 4, which in turn are fastened or otherwise engaged with the valve posts VP, this action directs the distal ends 4D of the post members 4 towards the axis X1, and with them the valve posts VP themselves.

As noted, the engagement of the thread T with the post members 4 occurs at the distal ends 4D in order to create an offset between the engagement location with the thread T and the connection location of the post members 4 to the annular portion 2, i.e. in order to bending moment capable of folding or flexing the post members 4 inwardly. Accordingly, in embodiments the engagement of the threads T with the post members 4 may occur further from the distal end than shown in the figures, provided that the resulting bending moment upon tensioning of the threads T remains high enough to overcome the bending resistance of the post members 4 (in embodiments, for instance, the elastic resistance of the sections 8 of wire like material).

Accordingly, in embodiments a storage kit may be provided that includes a container and the heart valve prosthesis P stored in the container.

In embodiments wherein the heart valve prosthesis P is stored with the post members 4 (and the valve posts VP) in the deployed condition—protrusion P1—the storage container may be a commonly used container such as a jar, possibly filled with a preservation solution. However, when the valve V is made of a polymer fabric (both the leaflets VL and the remainder of the valve V) or when the valve features one or more portions (typically the leaflets) made of a decellularized sterilized mammalian tissue having an extracellular matrix, wherein a plurality of interstitial spaces of the extracellular matrix include a solution of one or more polyols (so-called “dry” tissue, such as dry pericardium), the prosthesis P may be dry stored in the container.

In embodiments wherein the heart valve prosthesis P is stored with the post members 4 (and the valve posts VP) in the collapsed condition—protrusion P2—the storage container may be provided as a blister container, for instance comprising a first layer, a second layer joined to the first layer along a perimeter that defines a (blister) socket, and the prosthesis P arranged within the socket as a tablet. In this regard, when the post members 4 are moved to the collapsed position the entire valve V (and the prosthesis P accordingly) is itself collapsed in an essentially lenticular or disc-like shape which conveniently lends itself to storage in a blister socket. Again, if the material the valve V is made requires wet storage in a preservation solution, the socket can be filled with a preservation solution—in a manner per se known—while in embodiments allowing for a dry storage of the valve V the prosthesis P may be dry stored as a tablet proper, possibly even under vacuum. In this regard, note that the presence of the holder does not significantly impact the overall dimensions of the prosthesis P when the structure 1 has the post members 4 in the collapsed condition.

Whatever the storage solution, implantation of the heart valve prosthesis in embodiments begins with the supporting structure 1 in the collapsed condition. As noted, the essentially lenticular or disc-like shape of the prosthesis P overall achieves a significant advantage in that the surgical access may be reduced in size as compared to prior art implantations. To offer a comparison, the prosthesis P may be introduced into the patient's body for delivery in a way that at least roughly resembles the introduction of a coin through a coin slot.

Manipulation of the prosthesis P inside the body of the patient may be achieved by mating the holder 10 to a delivery instrument, possibly provided with steering and/or illumination facilities in a manner per se known.

In the exemplary circumstances of a mitral valve replacement, i.e. when the valve V is configured as a mitral valve prosthesis, the prosthesis P is approached to the implantation site in a proximal to distal direction in the reference system of the supporting structure 2. In other words, the prosthesis P is advanced through the body of the patient and to the implantation site “valve V first”, so to say (this may be generally true for other implantations—not limited to the mitral valve, depending on the surgical access).

Guidance of the prosthesis P through the body of the patient may be provided through parachuting threads as usual, and this is where the technical advantages of the supporting structure 1 come into play. The prosthesis P may in fact be advanced and positioned at the implantation site, together with all the routine adjustment and enlargement action of the implantation site through wobbling movements of the prosthesis P, without any risk of suture entrapment. This is due to the collapsed configuration of the post members 4 (and the valve posts VP accordingly, which thanks to a combination of the protrusion P2 (and, in the embodiments disclosed in the foregoing, the inward folding of the posts VP) essentially takes the posts VP away from the critical peripheral areas whereat suture entrapment may occur. The low protrusion P2 of the post members 4 also makes sure that even under the most critical circumstances the prosthesis P exhibits such a low profile that it becomes virtually impossible to get trapped into parachuting threads once the same are tied into sutures.

When implantation is completed, for instance with suturing of the prosthesis P at the sewing cuff SC (if present) or in general to the implantation site, the practitioner may take advantage of the cutoff sections of the threads S across the holes 10A and 10B and severe the threads T thereat, accordingly releasing the constraining action that keeps the post members 4—and the valve posts VP with them—collapsed into the lumen of the valve V. severing of the threads T in fact breaks continuity of the threads between the first ends and the second ends thereof, a condition which immediately releases the post members, provided that the engagement thereof to the threads T occurs between the first end and the second end, and the threads T can slip relative to the post members 4 through the holes 4H.

In this regard, having the cutoff sections of the threads T on an opposite side of the holder 10 than the supporting structure 1 presents the cutoff sections at a location still open to the action of the practitioner, as opposed to the other side of the holder 10 on which the already implanted valve V facing the confined enclosure of the left ventricle—making it impossible to provide further manipulation from there—stands.

Once the threads T are severed at the cutoff sections the holder 10 can be taken away of the body of the patient as the coupling thereof with the annular portion 2 is released.

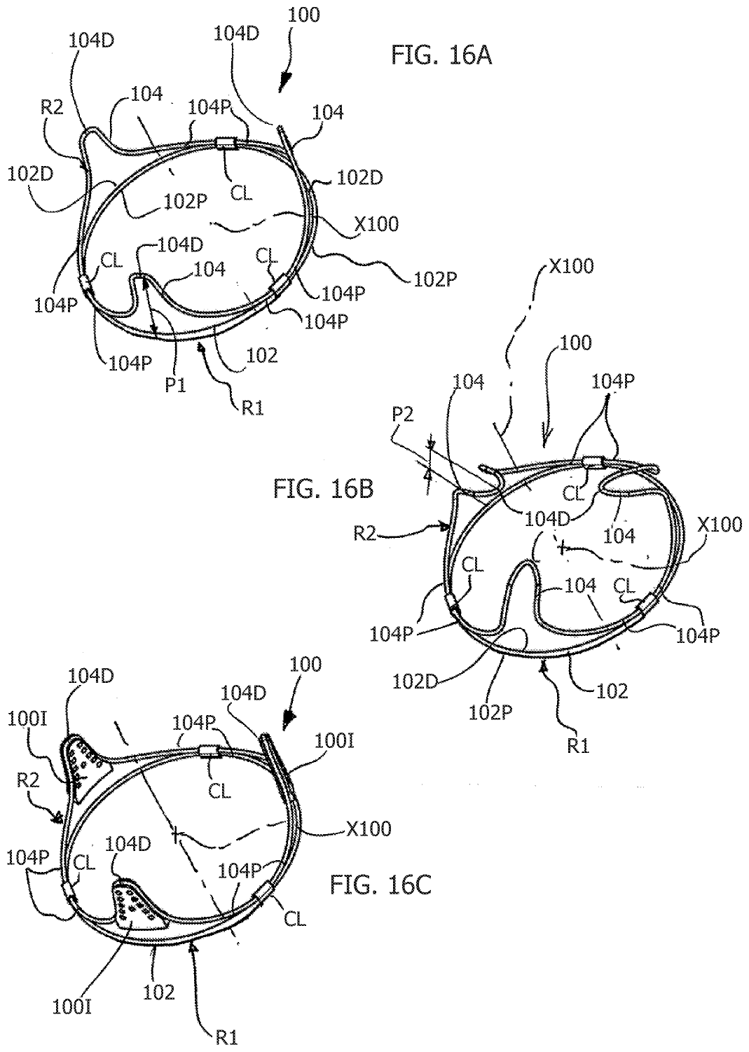

With reference to FIGS. 16A-16C, reference number 100 designates as a whole a supporting structure according to further embodiments. By way of general remark, reference numbers used within the context of the “100” embodiments are the same as those already used within the context of the “1” embodiments, only staggered by 100, and with identical meaning. For this reason, and for brevity, portions of the detailed description rendered in respect of the “1” embodiments may not be necessarily repeated for the “100” embodiments. As far as the functional description, what already provided in respect of the supporting structure 1 and the related prosthesis P and storage kit readily applies to the 100 embodiments, of course subject to implications due to the structural differences that will be noted in the following.

With reference to FIG. 16, in embodiments the supporting structure 100 includes:
an annular portion 102 defining a supporting structure longitudinal axis X100 (in other terms, the annular portion 102 includes a longitudinal axis X100 that defines the supporting structure longitudinal axis, i.e. the longitudinal axis of the supporting structure 100),
a plurality of post members 104 arranged along the annular portion 102 around the supporting structure longitudinal axis X100, wherein each post member 104 extends from a (twin) proximal end 104P to a distal end 104D, the distal 104D end having a protrusion with respect to the annular portion 102 in a direction parallel to the axis X100. Each post member 104 is movable with respect to the annular portion 102 so as to vary the protrusion of the distal end 104D thereof from a first protrusion P1 (FIG. 16A) to a second protrusion P2 (FIG. 16B), lower than the first protrusion P1.

The drive member operatively connected to the post members 104 and operable to move the post members 104 from the first protrusion P1 to the second protrusion P2 is not shown herein for clarity, as it may be configured as the drive member 6 already disclosed in the foregoing. The same applies to the use of the threads T and to the holder 10 and all of the related arrangements (e.g. tying of the threads T and so). All of the disclosure regarding the holder 10 and the drive member 6 in the embodiments per the foregoing applies.

In embodiments, the annular portion 102 may be configured to fit at the prosthetic valve annulus VA, and each of the post members 104 may be configured to fit at a corresponding valve post VP of the heart valve prosthesis V. In embodiments, the supporting structure 100 may include three post members 104, and the prosthetic valve V may include three valve posts VP (and three valve leaflets VL) accordingly.

In embodiments, the supporting structure 100 comprises:
a first ring R1, made of a first wire like element, wherein the annular portion 102 comprises the ring R1,
a second ring R2 made of a second wire-like element and defining the post members 4. To this end, the second ring is shaped so as to exhibit a plurality of V-shaped protrusions each defining an individual post member 104 extending axially of the ring R2: this also results in the post members having twin proximal ends 104P.

In embodiments, the ring R1 and the ring R2 are coupled together to define the supporting structure 100, for instance by clipper members CL arranged along the rings R1, R2.

In the embodiments of FIG. 16C each V-shaped protrusion comprises an insert 100I at a portion comprised between adjacent sections of each V-shaped protrusion. This results in post members 104 comprising wire-like sections from the ring R2 and the insert 100I, the latter possibly provided with a pattern of sewing holes for securing the valve posts VP thereto.

As regards the protrusions P1, P2, in embodiments the same result in an overall axial length of the prosthesis measured along the axis X100 which varies consistently with the protrusion, whereby the protrusion P1 is associated to a first axial length, and the protrusion P2 is associated to a second axial length, smaller than the first axial length.

Protrusions P1, P2 are measured between the end(s) 104D and a distal end 102D of the annular portion 102.

The axial length is measured between the distal end(s) 104D and a proximal end 102P of the annular portion. It can be said that protrusions P1 and P2 are identical to the axial length of the supporting structure, minus the thickness/diameter of the wire-like member of the ring R1.

As to the protrusions P1, P2, again they may be regarded as an indication of an axial extension of the post members 104 off the annular portion 102. In general terms, the condition corresponding to the protrusion P1 may be regarded as a “deployed” condition of the post members 104, while the condition corresponding to the protrusion P2 may be regarded as a “collapsed” condition of the post members 104. Reference to the “deployed” and the “collapsed” condition will be made throughout the disclosure of the embodiments 100 in the same manner as above.

As far as the routing of the threads T, although not shown in detail, in such embodiments they generally do not run through holes in the post members 104, as the wire-like construction thereof would hardly allow such provision, but they engage (and slide along) a notch at the distal end 104D of the post members 104, whereby tensioning of the threads T moves the post members 104 from the deployed condition to the collapsed condition by “pushing” the post members 104 proximally.

However, in case the inserts 100I are provided, a through hole for routing the threads T may be envisaged, thereby resulting in an overall operation not different from that of the supporting structure 1.

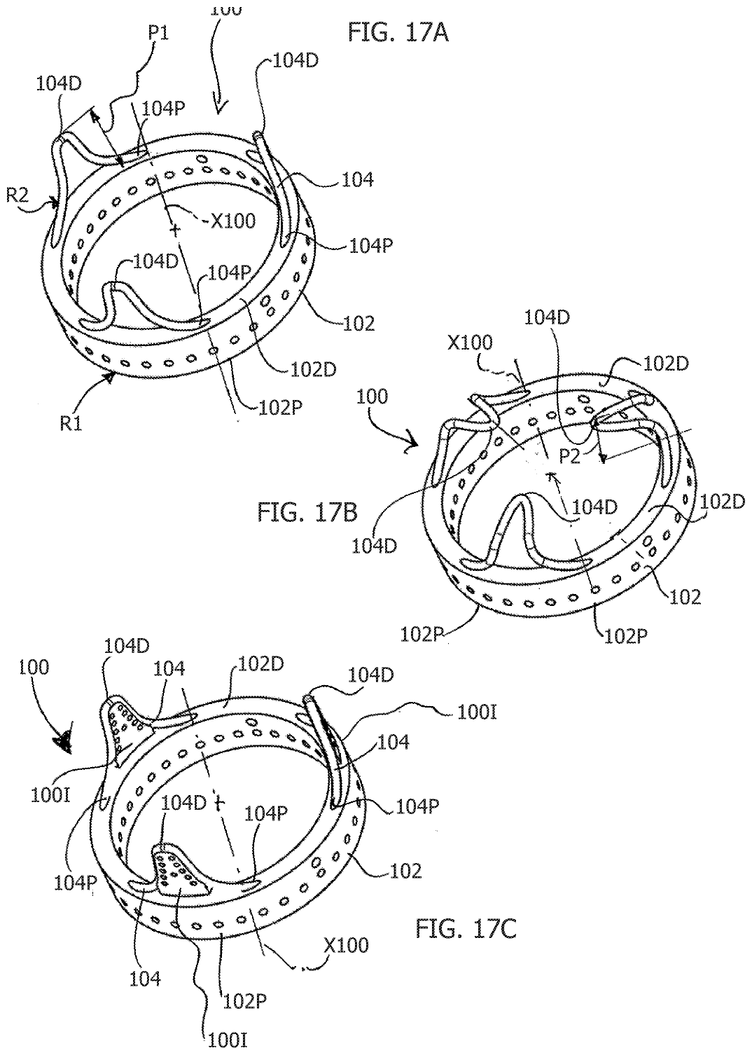

With reference to FIGS. 17A-17C (equivalent to FIGS. 16A-16C), in further embodiments of the supporting structure 100 the ring R1 is not made of a wire-like element but it is embodied as a collar-like member enclosing the second ring R2, while leaving the V-shaped protrusions making up the post members 104 exposed. Taking advantage of the increased area and volume of the ring R1 as a collar-like member, sewing holes R1H may be conveniently provided facilitating the coupling of the valve V (at the annulus VA) to the annular portion 102 comprising the ring R2. The remainder is otherwise unchanged as compared to the embodiments of FIGS. 16A to 16C, including the provision of the inserts 100I.

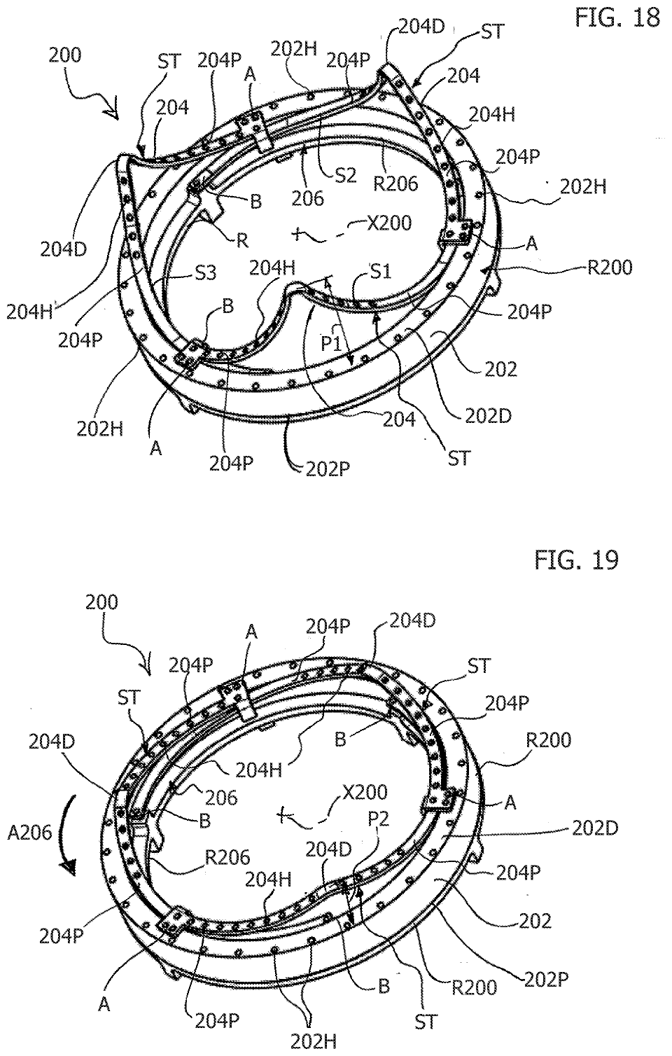

With reference to FIGS. 18 and 19, reference number 200 designates as a whole a supporting structure according to yet further embodiments. By way of general remark, reference numbers used within the context of the “200” embodiments are the same as those already used within the context of the “1” embodiments, only staggered by 200, and with identical meaning. For this reason, and for brevity, portions of the detailed description rendered in respect of the “1” embodiments may not be necessarily repeated for the “200” embodiments. As far as the functional description, what already provided in respect of the supporting structure 1 and the related prosthesis P and storage kit readily applies to the 200 embodiments, of course subject to implications due to the structural differences that will be noted in the following.

With reference to FIGS. 18, 19, in embodiments the supporting structure 200 includes:
an annular portion 202 defining a supporting structure longitudinal axis X200 (in other terms, the annular portion 202 includes a longitudinal axis X200 that defines the supporting structure longitudinal axis, i.e. the longitudinal axis of the supporting structure 200,
a plurality of post members 204 arranged along the annular portion 202 around the supporting structure longitudinal axis X300, wherein each post member 104 extends from a (twin and variably positioned) proximal end 204P to a distal end 204D, the distal 204D end having a protrusion with respect to the annular portion 202 in a direction parallel to the axis X200. Each post member 204 is movable with respect to the annular portion 202 so as to vary the protrusion of the distal end 204D thereof from a first protrusion P1 (FIG. 18) to a second protrusion P2 (FIG. 19), lower than the first protrusion P1.

In embodiments, the annular portion 202 may be configured to fit at the prosthetic valve annulus VA, and each of the post members 204 may be configured to fit at a corresponding valve post VP of the heart valve prosthesis V. In embodiments, the supporting structure 200 may include three post members 204, and the prosthetic valve V may include three valve posts VP (and three valve leaflets VL) accordingly.

In embodiments according to FIGS. 18 and 19, the supporting structure 200 comprises:
a first ring R200 coaxial to the supporting structure longitudinal axis X200, wherein the annular portion 202 comprises the first ring,
a plurality of deformable struts ST arranged circumferentially along the ring R200, each of the deformable struts ST having a first A end fixed to the ring R200, and a second end B fixed to a drive member 206. Each deformable strut ST buckles distally of the first ring R200 between ends A and B to define a V-shaped protrusion providing a respective one of said post members 204.

The drive member 206 is configured to vary a circumferential distance between the first end A and the second end B of each deformable strut ST from a first distance to a second, lower distance. As the extent by which the deformable struts ST buckle distally of the ring R200 depends on the circumferential distance concerned, variation of the circumferential distance between ends A, B results in the struts ST defining the protrusion P1 (FIG. 18) or the protrusion P2 (FIG. 19). In other words, movement of the post members 204 relative to the annular portion 200 occurs via a transition from a first distance between ends A, B to a second distance between ends A, B, wherein the second distance is bigger than the first distance. In fact, smaller inter ends (A to B) distances result in a larger buckling of the struts ST, and more protrusion from the annular portion 200, while larger inter-ends (A to B) distances result in a lesser buckling of the struts ST and less protrusion from the annular portion 200.

In embodiments, the drive member 206 comprises a second ring R206 rotatable around the supporting structure longitudinal axis X200 relative to the first ring R200. The second ring 206 may be rotatably coupled to the first ring R200, and coupled thereto via the very struts ST, whose ends B are fastened to the ring 206. Rotation A206 of the ring R206 relative to the ring R200 results in an increase of inter ends distance and transition from the condition of protrusion P1 to the condition of protrusion P2 for the post members 204 defined by the struts ST. an opposite rotation restores the condition of FIG. 18.

It will be appreciated that the drive member 206 is capable of bidirectional transition between protrusions P1 and P2, simply by reversing the direction of rotation of the ring R206 around the axis X200.

Note that ends A and B provide the proximal ends of the post members 204, and in these embodiments the “B” proximal end lies more proximally than a distal end 202D of the annular portion 202/ring 200.

As regards the protrusions P1, P2, in embodiments the same result in an overall axial length of the prosthesis measured along the axis X200 which varies consistently with the protrusion, whereby the protrusion P1 is associated to a first axial length, and the protrusion P2 is associated to a second axial length, smaller than the first axial length.

Protrusions P1, P2 are measured between the end(s) 204D and the distal end 202D of the annular portion 102. The axial length is measured between the distal end(s) 204D and a proximal end 202P of the annular portion.

As to the protrusions P1, P2, again they may be regarded as an indication of an axial extension of the post members 204 off the annular portion 202. In general terms, the condition corresponding to the protrusion P1 may be regarded as a “deployed” condition of the post members 204, while the condition corresponding to the protrusion P2 may be regarded as a “collapsed” condition of the post members 204.

Unlike the 1 and the 100 embodiments, a holder may be coupled to the annular portion 202 for manipulation of the prosthesis P in the body of the patient, but it does not serve to support the drive member 206, nor does it serve to provide cutoff locations for threads. To this end, the deployment/collapsing action of the post members 204 does not depend on the tensioning of threads T, but only on the rotation of the ring making up the drive member 206. Accordingly, in such embodiments no threads T may be required to manipulate the post members 204. Threads may be required, however, to temporarily secure the holder to the annular portion 202 for manipulation of the prosthesis P within the body of the patient. Such threads can then be severed to release the holder from the prosthesis P.

Additionally, fitting of the valve V onto the supporting structure 200 to provide the prosthesis 1 may involve different coupling solutions than those disclosed in the foregoing. For instance, while the annular portion 202 may be provided with a circular array of sewing holes 202H for suturing the valve V at the annulus VA, the struts ST may be provided as ribbon like members including a plurality of sewing holes 204H. as can be appreciated from FIGS. 18 and 19, the relative position of groups of sewing holes 204H varies during transition from the deployed condition to the collapsed condition, whereby valves V that can be mounted on the supporting structure 1 include valves wherein the valvular sleeve is made of a stretchable fabric to accommodate the deformation of the post members 204. The leaflets VL may be made of any of the above disclosed materials, essentially in that they experience less deformation.

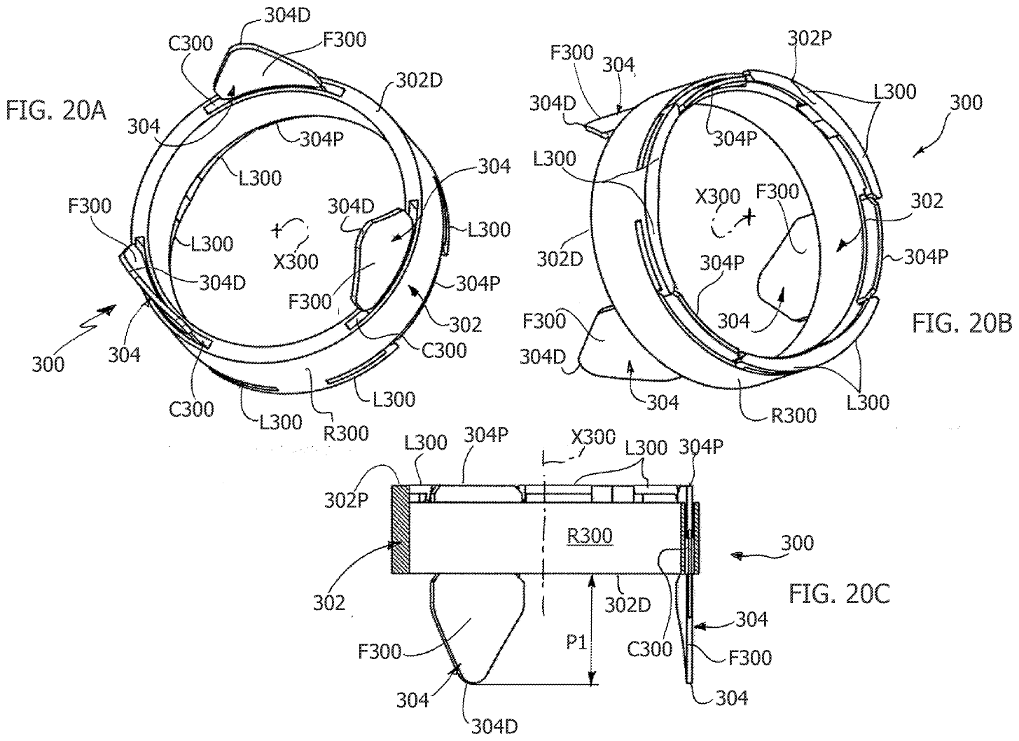

With reference to FIGS. 20A to 23D, reference number 300 designates as a whole a supporting structure according to yet further embodiments. By way of general remark, reference numbers used within the context of the “300” embodiments are the same as those already used within the context of the “1” embodiments, only staggered by 300, and with identical meaning. For this reason, and for brevity, portions of the detailed description rendered in respect of the “1” embodiments may not be necessarily repeated for the “300” embodiments. As far as the functional description, what already provided in respect of the supporting structure 1 and the related prosthesis P and storage kit readily applies to the 300 embodiments, of course subject to implications due to the structural differences that will be noted in the following.

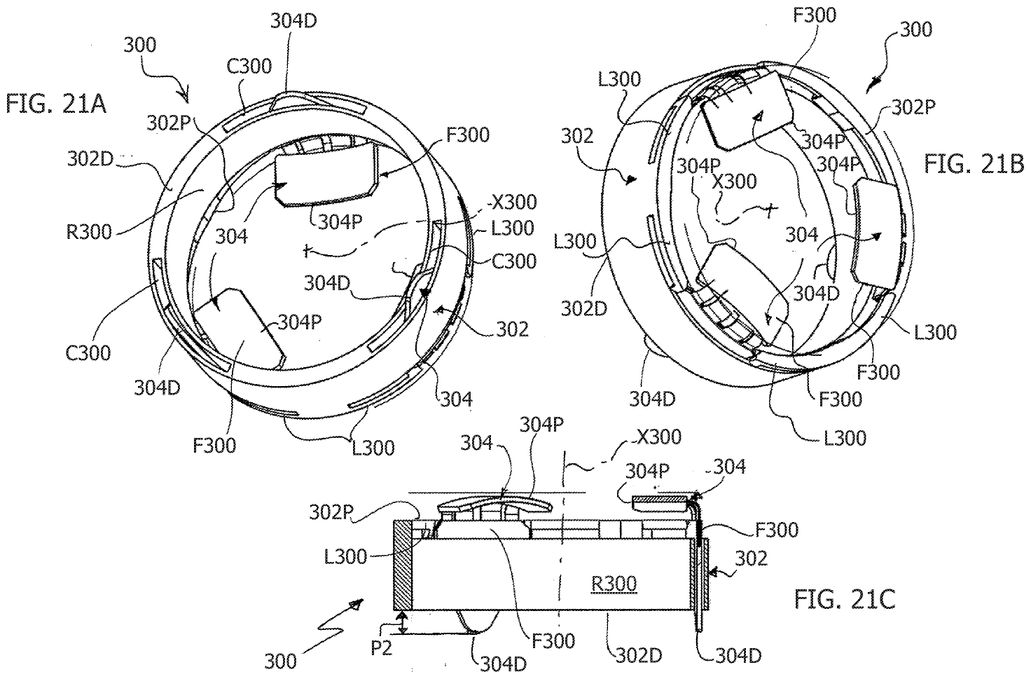

With reference to FIGS. 20A-C, 21A-C, in embodiments the supporting structure 300 includes:
an annular portion 302 defining a supporting structure longitudinal axis X300 (in other terms, the annular portion 302 includes a longitudinal axis X300 that defines the supporting structure longitudinal axis, i.e. the longitudinal axis of the supporting structure 300),
a plurality of post members 304 arranged along the annular portion 302 around the supporting structure longitudinal axis X300, wherein each post member 304 extends from a proximal end 304P to a distal end 304D, the distal end 304D having a protrusion with respect to the annular portion 302 in a direction parallel to the axis X300. Each post member 304 is movable with respect to the annular portion 302 so as to vary the protrusion of the distal end 304D thereof from a first protrusion P1 (FIG. 20C) to a second protrusion P2 (FIG. 21C), lower than the first protrusion P1.

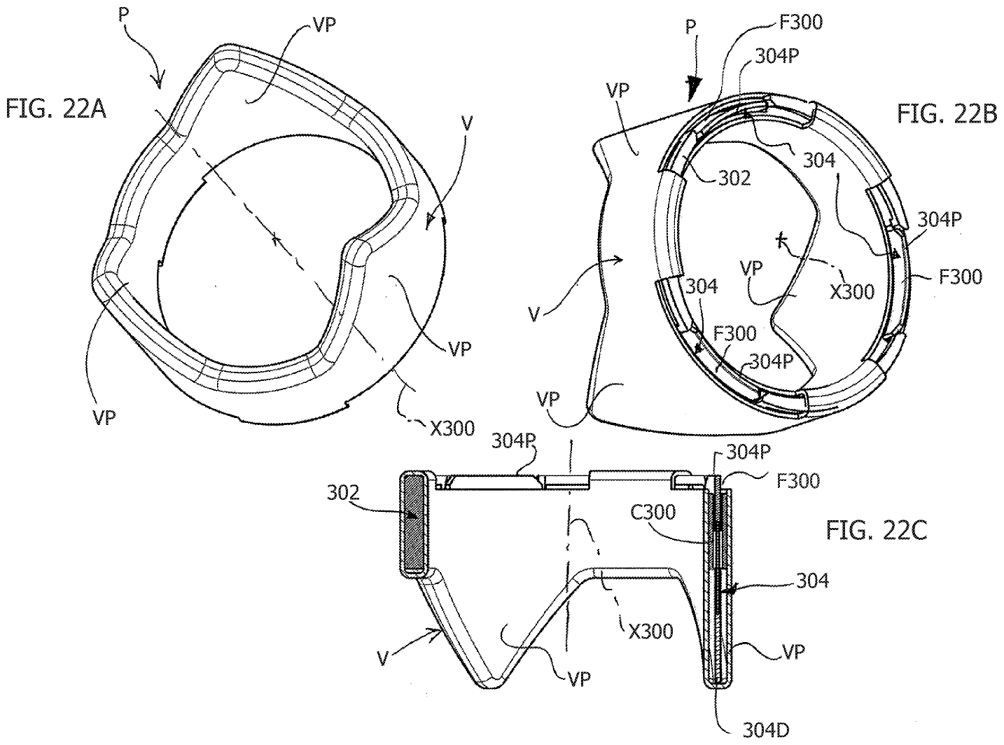

In embodiments, the annular portion 302 may be configured to fit at the prosthetic valve annulus VA, and each of the post members 304 may be configured to fit at a corresponding valve post VP of the heart valve prosthesis V (FIGS. 22A-C, 23A-C).

In embodiments, the supporting structure 300 may include three post members 304, and the prosthetic valve V may include three valve posts VP (and three valve leaflets VL) accordingly.

In embodiments, the supporting structure 300 comprises:
a hollow ring R300, wherein the annular portion 302 comprising the hollow ring R300,
a plurality of finger-like members F300 arranged circumferentially along the hollow ring R300 and around the supporting structure longitudinal axis X300, each finger-like member F300 comprising a proximal end 304P and a distal end 304D, and defining a respective support post 304 (the ends 304P and 304D are the distal ends of the post member 304 as well).

The hollow ring R300 comprises one or more cavities C300, preferably one for each post member 304, extending axially and through which each finger-like member F300 is axially movable to define the first protrusion P1 and the second protrusion P2 of the post members.

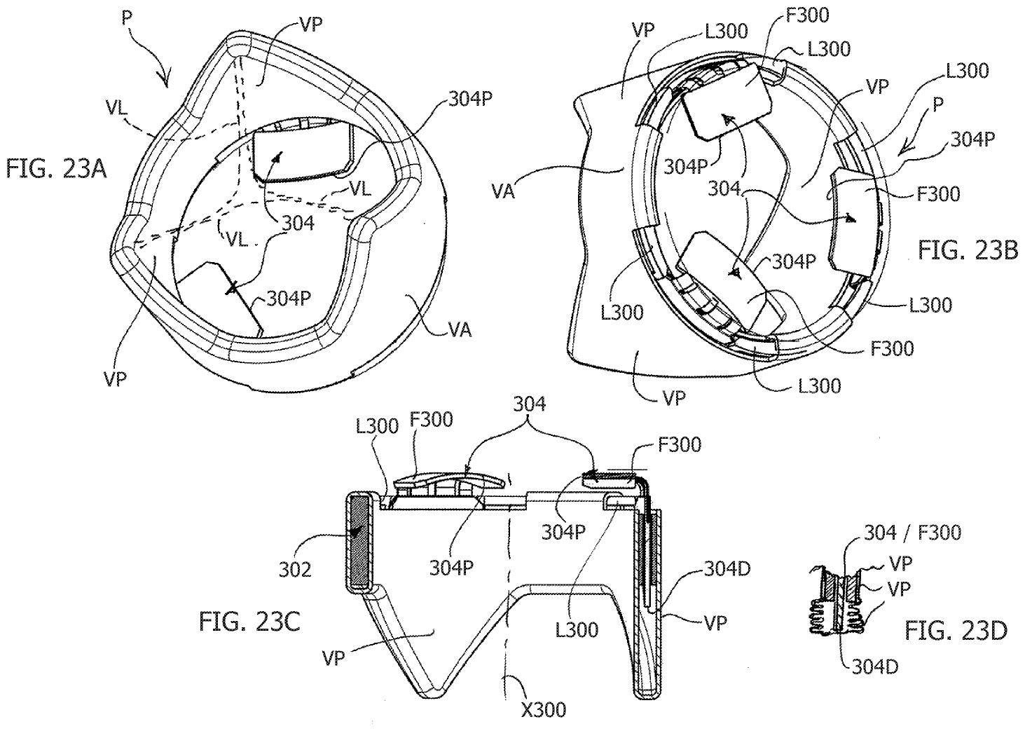

In embodiments, each finger-like member F300 is V-shaped in an undeformed condition (FIGS. 21A-21C, 23A-23D), associated to the collapsed configuration. In such embodiments, each finger-like member comprises a proximal portion and a distal portion hinged to one another to define the V-shape. An integral hinge is generally provided between the proximal portion and the distal portion of the finger-like members F300. The V-shape may be achieved by making the finger-like members F300 as shape memory elements, i.e. made of a shape memory material.

In other words, when the post members 304 have the second protrusion P2 the finger-like members F300 are retracted into the one or more cavities C300 of the hollow ring R300 in the undeformed condition, with a (proximal) portion protruding radially inwardly of the hollow ring R300. When, instead, the post members 304 have the first protrusion P1 the finger-like members F300 are extracted distally of the hollow ring R300, and are straightened into a substantially linear pattern by the walls of the respective cavity C300.

As regards the protrusions P1, P2, in embodiments the same result in an overall axial length of the prosthesis measured along the axis X300 which varies consistently with the protrusion, whereby the protrusion P1 is associated to a first axial length, and the protrusion P2 is associated to a second axial length, smaller than the first axial length.

Protrusions P1, P2 are measured between the end(s) 204D and a distal end 302D of the annular portion 302/ring 300. The axial length is measured between the distal end(s) 304D and a proximal end 302P of the annular portion.

As to the protrusions P1, P2, again they may be regarded as an indication of an axial extension of the post members 304 off the annular portion 302. In general terms, the condition corresponding to the protrusion P1 may be regarded as a “deployed” condition of the post members 304, while the condition corresponding to the protrusion P2 may be regarded as a “collapsed” condition of the post members 304.

Advantageously, in embodiments the hollow ring R300 may comprise one or more locking features L300 configured to engage the finger-like members F300 when the post members have the first protrusion to lock the finger-like members in position. Such locking features may for instance comprise cantilevered struts at the proximal end of the annular portion 302 providing an axial shoulder to the finger-like members F300 as soon as the latter are extracted enough to reach the first protrusion P1.

With reference to FIGS. 22A-C, 23A-C, a prosthesis P comprising the valve V mounted on the supporting structure 300 will now be disclosed (valve leaflets VL are only shown in dashed lines in FIG. 23 to avoid obstructing the view to other components).

The supporting structure 300 is generally configured as a core to the valve V in the prosthesis P. to this end, with reference to the sectional views of FIGS. 22C, 23C, the finger-like members F300/post members 304 are arranged inside valve posts VP shaped as pockets, whereby the extraction/retraction of the finger-like members F300/post members 304 relative to the annular portion 302 results in an extension/collapse of the pockets making up the valve posts VP. In such embodiments, similarly to the prosthesis P comprising the supporting structure 200, there is no inward folding of the post members 304 and the valve posts VP when transitioning from the deployed condition to the collapsed condition, but rather an axial collapse of the valve posts VP, for instance achieved via a mechanical axial coupling (i.e. effective in an axial motion) between the finger-like members F300/post members 304 in an axial motion.

In this regard, the sectional view of FIG. 23C does not show collapsed valve posts VP to avoid complicating the figure, but a partial sectional view thereof is offered in FIG. 23D. Collapse is reversible, in that when the post members 304/finger-like members F300 are operated to restore the deployed condition and the protrusion P1, the valve posts VP are extended back into the deployed shape visible in FIG. 22C by the axial motion of the finger-like members F300 through the cavities C300.

The implantation of the prosthesis P is otherwise unchanged, but—similarly to the prostheses P including the supporting structure 200, the valvular sleeve of the valve V is generally made of a stretchable elastic material to better accommodate the resulting deformations.

With reference to FIGS. 24 to 28, embodiments of a holder 310 also including a drive member 316 for controlling the movement of the finger-like members F300 making up the post members 304 will now be disclosed.

The holder 310 comprises a first member 310A, a second member 310B, and a third member 310C. All of the members 310A-C are coaxial to each other and to an axis X310 coaxial to the axis X300 of the supporting structure 300.

In embodiments, each of the members 310A, 310B, 310C has a spoke wise configuration, i.e. it includes a hub coaxial to the axis X310 and a plurality of spokes extending radially from the hub.

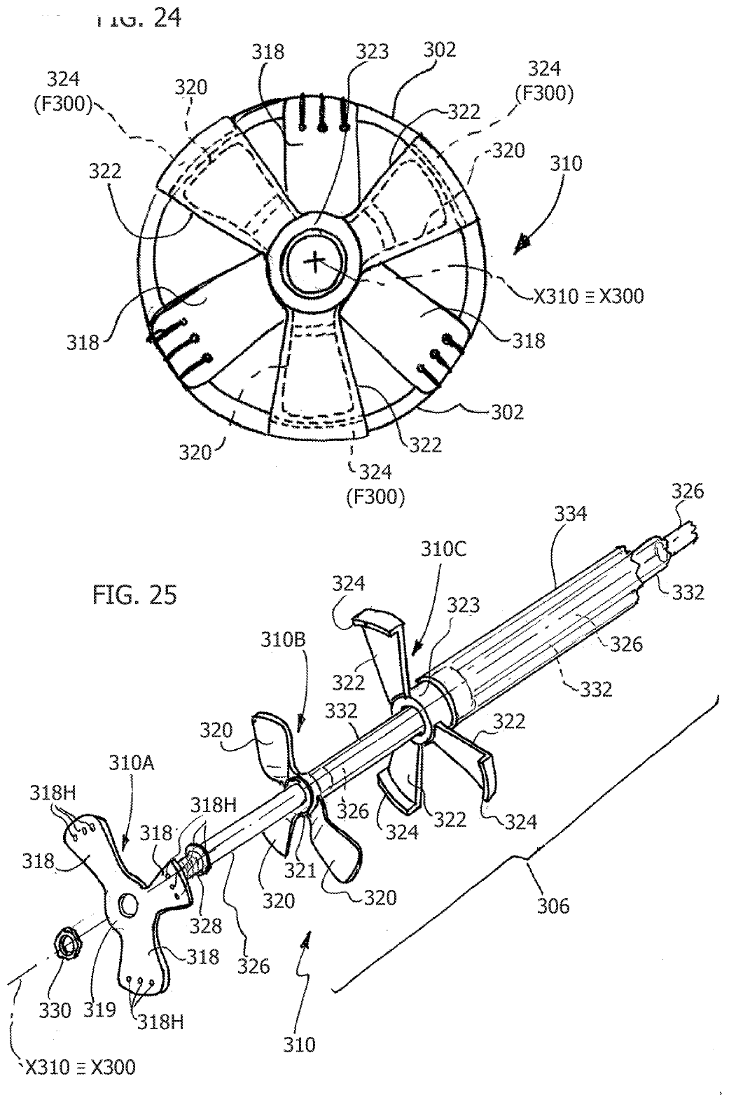

In detail, FIG. 25, in embodiments the first member 310A may include three spokes 318 extending radially from a hub 319. The spokes 318 may be provided in principle in a different number, while the embodiment shown herein relates to the supporting structure 300 featuring three post members 304 as shown in the figures. In such configuration, the three spokes may be arranger 120 degrees apart around the axis X310.

At the end of each spoke opposite to the hub 319 one or more sewing holes 318H are provided to accommodate thread-like attachments of the member 310A to the annular portion 302. In general, as the member 310A is fixed to the annular portion 302 (and is fixed relative to the supporting structure 1 overall), other attachment methods may be envisaged.

The second member 310B may include three spokes 320 extending radially from a hub 321. The spokes 318 are provided in a number equal to the number of the post members 304, as each spoke is operatively associated to one post member 304/finger element F300. In embodiments, the spokes 320 may extend at least partially in the axial direction, whereby—notwithstanding the essentially radial protrusion with respect to the hub 321, the spokes 320 also have a protrusion in the axial direction X300. Spokes 320 have a smaller radial extension than spokes 318, as spokes 320 are intended to fit within the inner diameter of the annular portion 302 to interact with the finger-like members F300 as will be disclosed in the following. In other terms, the diameter of member 310B is smaller than the diameter of member 310A, and is generally smaller than an inner diameter associated to the inner surfaces of the finger-like members F300.

Similarly to the member 310B, the third member 310C may include three spokes 322 extending radially from a hub 323. The spokes 322 are provided in a number equal to the number of the post members 304 (and equal to the number of the spokes 320), as each spoke 322 is operatively associated to one post member 304/finger element F300. In embodiments, the spokes 320 may extend at least partially in the axial direction by means of axial extensions 324 located at the end of the spoke 322 opposite to the hub 323. The spokes 322 have a higher radial extension than spokes 320, as spokes 322 are intended to radially overlap the proximal ends of the post members 304 to exert an axial thrust as will be detailed in the following.

In other terms, the diameter of member 310C is smaller than the diameter of member 310B.

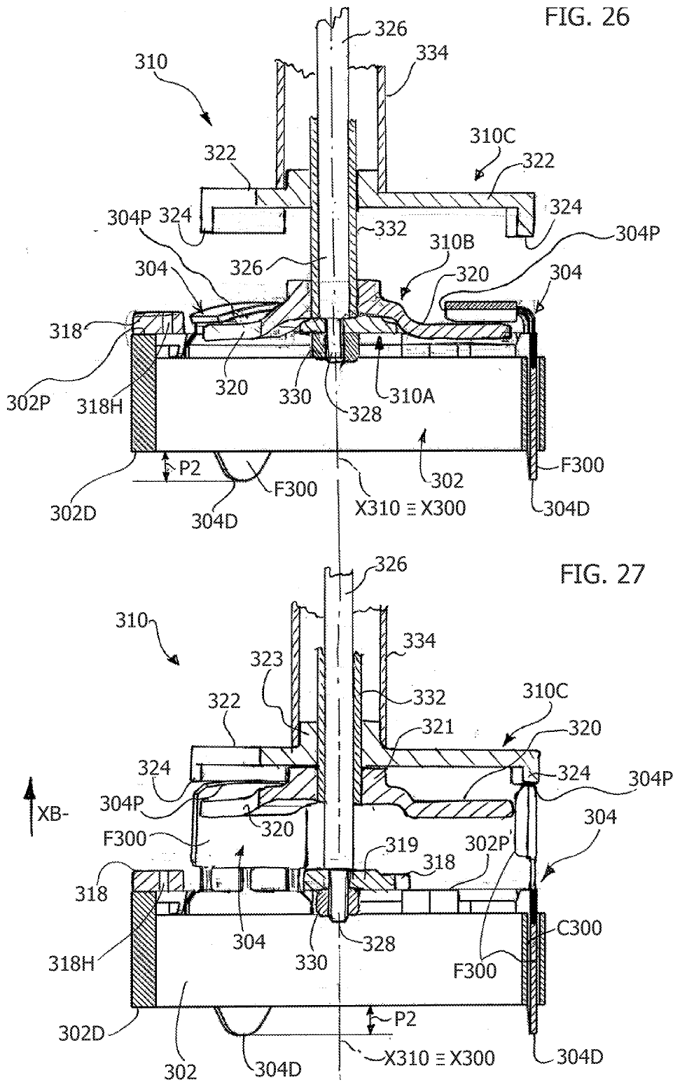

With reference to FIGS. 24 and 25, the arrangement of members 310A, 310B and 310C is such that member 310A is fixed to the annular portion 302 at the proximal end 302P, member 310B is arranged adjacent to the member 310A and on the proximal side thereof, while member 310C is again arranged next to member 310B further shifted in the proximal direction, whereby member 310B is comprised between members 310A and 310C. in an initial, pre-operative configuration shown in FIG. 26, member 310C is axially spaced from members 310B and 310A, which may be arranged essentially in axial contact with one another on in any case with a lesser axial spacing.

Additionally, while members 310B and 310C are arranged angularly aligned as they are both configured to operate on the finger-like members F300/post members 304, member 310A is angularly staggered (in these three-spoke embodiments by 60 degrees) to attach to location in between subsequent post members 304 and to avoid interfering with the operation of members 310B and 310C.

With reference to FIG. 25, in embodiments a core shaft 326 coaxial to the axis X310 is coupled to the first member 310A, for instance by means of an axial shoulder on the shaft 310A and a threaded stud 328 traversing the hub 319 and locked in position by a nut 330. Other attachment methods, for instance interference fit may anyway be envisaged. Furthermore, in embodiments the core shaft 326 may not necessarily be a rigid member, and may feature some steering capabilities—for instance at the section “upstream” (or proximal) of the member 310C (which may be kept straight to facilitate axial movement of members 310B, 310C) to orient the complex of the members 310A, 310B and 310C, and the prosthesis attached thereto, at an angle to the axis X310, for instance to cope with surgical access constraints or to facilitate insertion of the prosthesis P through a smaller surgical access.

In embodiments, the hub 321 slidingly fits onto the core shaft 326 so to be movable axially along the axis X310, using the core shaft 326 as a guide, and is coupled to an intermediate hollow shaft or sheath 332 through which axial sliding of member 310B along the axis X310 may be controlled.

In embodiments, the hub 323 in turn slidingly fits onto the intermediate hollow shaft or sheath 332 so to be movable axially along the axis X310, using the shaft 332 as a guide, and is coupled to an outer hollow shaft or sheath 334 through which axial sliding of member 310C along the axis X310 may be controlled.

The complex of members 310B and 310C may functionally be regarded as a drive member 316 for the post members 304.

With reference to FIGS. 26 to 28, operation of the holder 310 in connection with variation of the protrusion of the post members 304 will now be disclosed.

FIG. 26 is representative of an initial or pre-operative condition which precedes the introduction of the prosthesis P into the body of the patient. This condition may be provided as a factory condition, wherein prosthesis P comes packaged in the “collapsed” condition of FIG. 26 together with the holder 310, or may be achieved directly in the operational field.

Either way, the holder 310 is assembled to the supporting structure 300 such that the spokes 318 are attached to inter-post locations and the spokes 320 are overlapped by folded proximal portions of the members F300, whereby any axial movement of the member 310B in the proximal direction is “countered” by the folded portions of the finger-like members F300.

This condition may be achieved by providing the prosthesis P with the valve posts VP in the deployed condition, then attaching the member 310A to the annular portion 302 (this may be done even in the operational field, for instance when the prosthesis P is packaged in the deployed condition without an attached holder 310, hence without a pre-attached drive member 316) by suturing the member 310A via the holes 318H), and manually driving the post members 304/F300 proximally into the cavities to shift from protrusion P1 to protrusion P2, which “folds” the proximal portions of the finger-like members F300 into overlap with the spokes 320. In embodiments, the holder 310 may at least partly stored with the prosthesis P in the collapsed condition: in such cases, the core shaft 326 and the intermediate shaft 332 may be split in two section at the axial location comprised between the members 310C and 310B, to leave only the members 310A and 310B attached to the prosthesis P/supporting structure 300 together with the terminal (distal) section of the shafts 326 and 332. The proximal sections of the shafts 326 and 332, together with the shaft 334 and the member 310C may then be coupled to the distal sections e.g. by snap fitting or threaded fitting to reassemble the holder 310 as it stands in FIGS. 25 through 28. This allows i.a. compact packaging of the prosthesis P (for instance in the blister container referred to above) and may relieve the practitioner in the operational field from the burden of attaching the holder 310 to the prosthesis P.

The prosthesis P is introduced into the body of the patient in the condition of FIG. 26, possibly with some degree of “steering” of the complex of elements 310A-C from the axis X300 as per the above. The prosthesis P reaches the implantation site through conventional surgical techniques and may be positioned through a wobbling motion to enlarge the implantation site as much as possible to reduce the pressure drop across the same, without the risk of suture entrapment as the prosthesis P features valve posts VP in the collapsed condition with protrusion P2. The prosthesis may be then sutured in place once positioning is completed.

From here, FIG. 27, a proximal axial motion of the shaft 332 is controlled so to provide a proximal axial translation XB− of member 310B. As visible in FIG. 27, this straightens the finger-like members from the resting L-shape thereof to an essentially rectilinear shape that is compatible with the sliding thereof along the cavities C300. Note that member 310C is generally static during this step, and it may essentially be used as a stop member for the axial travel of the member 310B. in this regard, axial spacing of member 310C from member 310B is generally chosen so that the former does not interfere with the straightening of finger-like members 304 during axial movement XB− of member 310B (radial interference is excluded anyway due to the difference in diameters referred to in the foregoing).

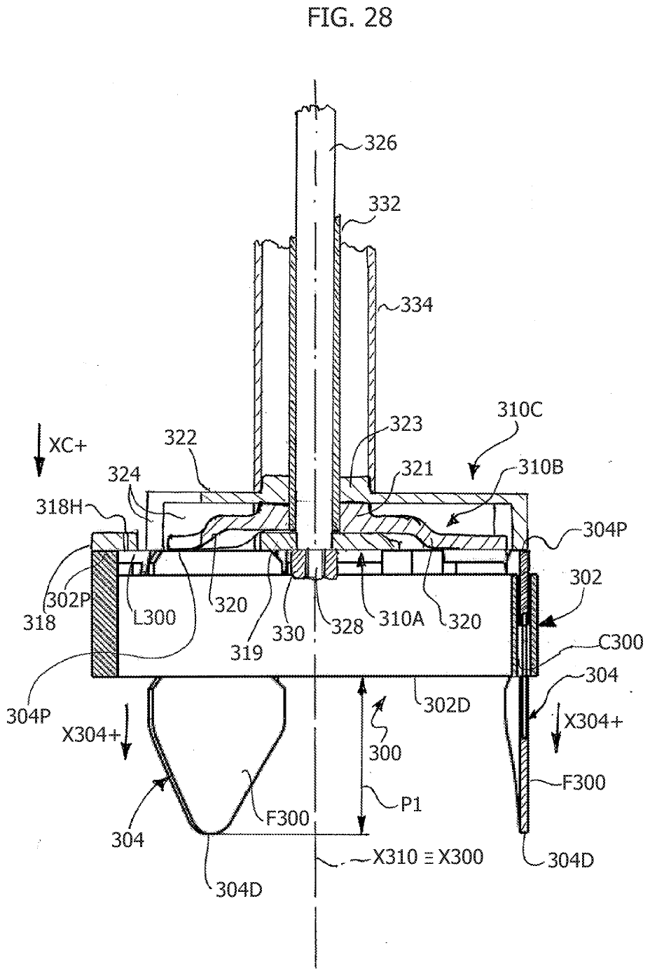

Next, FIG. 28, a distal axial movement XC+ of member 310C is controlled to exert an axial thrust via the spokes 322 (in embodiments herein via the extensions 324) onto the proximal ends of the post members 304/finger-like members F300. This pushes the straightened finger-like members F300 through the cavities C300 to achieve the first protrusion P1, and restore the working, deployed, shape of the prosthesis P. finger-like members F300 are anyway prevented from folding back into an L-shape by the very sliding through the cavities C300 and by the radial counter action of spokes 320, in view of the fact that member 310B is drawn into the axial movement XC+ by member 310C.

The extensions 324 may be chosen to have an axial length sufficient to cover the axial excursion between the hub 323 and the proximal end 302P so that upon completing the travel XC+ the proximal ends 304P are made to snap fit or otherwise interlock with the locking features L300, hence fixing the post members in the deployed condition with the protrusion P1.

Cutting of the threads bonding the member 310A to the annular portion 302 (or defeating of the attachment method alternative to the threads) breaks the holder free of the prosthesis P, which remains at the implantation site.

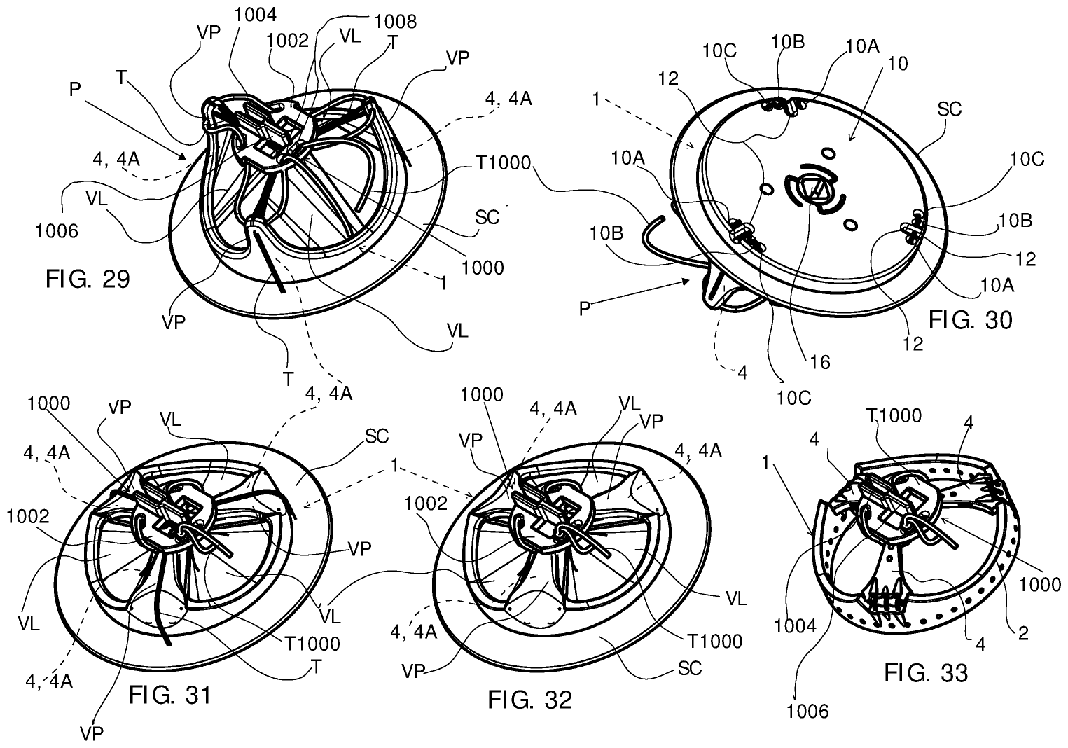

With reference to FIGS. 29 to 33, 34 to 37 and 38 to 42, embodiments will be disclosed featuring facilities to assist with aortic implantation of the prosthesis P, particularly with reference to the prostheses P including the support structure 1, 1A, 1B, as well as the support structure 100.

By way of general introduction, in minimally invasive aortic implantation procedures the holder 10, which serves as an attachment for the thread T routed through the post members 4, 4A, and the drive member 6, may become inaccessible when not removed prior to positioning the valve at the implantation site. However, removal of the holder 10 prior to finalizing the implantation has the undesired effect that the post members 4, 4A are released to the deployed position with the protrusion P1.

The embodiments of the figure sets referred to above each provide a solution to separate the holder 10 from the prosthesis P without releasing the post members 4, 4A to the protrusion to the deployed position with the protrusion P1. Overall, FIGS. 29 to 33, 34 to 37 and 38 to 42 all disclose embodiments of post member retention systems 1000, 2000, 3000 configured to operate in a staged fashion with respect to the drive member 6, i.e. the drive member 6 is operated first to set the post members to the second protrusion, then the post member retention systems 1000, 2000, 3000 is set up.

With reference to FIGS. 29 to 33, reference P designates as a whole the prosthesis P with the thread T already routed through the post members 4, 4A (depending on the embodiment of the support structure 1) and through the drive member 6, ready for operation of the latter. Routing may occur through the holes 4H or—as suggested by FIGS. 29 to 33—additional holes extending transverse to the holes 4H may be provided at the distal ends of the post members 4, 4A for this purpose. Such holes may intersect holes 4D or be provided at a separate location.

Reference number 1000 designates as a whole a first post member retention system configured to operate with the prosthesis P. The post retention system 1000 includes a retention facility 1002, and a retention thread T1000. The combination of a retention facility and a retention thread is common to all the embodiments herein.

The retention facility 1002 (FIG. 29) is provided essentially as a retention tab, and includes a cutoff portion 1004, a grasping rib 106—on opposite sides of which apertures are formed which facilitate grasping by a surgical clamp—and tie-up holes 1008. The retention thread T1000 is routed through the distal ends 4D of the post members 4, 4A (e.g. through the holes 4H) in an essentially triangular pattern with the following features:
the thread T1000 is configured to be tensioned with the post members 4 already collapsed from the protrusion P2 to the protrusion P1 by the drive member 6,
a section of the thread T1000 runs across the cutoff portion 1004 and is weaved through holes located on opposite sides of the cutoff portion 1004, whereby the latter—which is slot shaped—overlaps a section of the tensioned thread T1000 (here located on the hidden side of the facility 1002 facing the leaflets VL), to guide the practitioner in severing the section of the tensioned thread T1000 concerned;
free ends of the thread T1000 are tied to one another at the tying holes 1008 after tensioning the thread T1000, as visible in FIG. 31.

Under the above circumstances, severing of the thread T at the radial notches 12 as disclosed in the foregoing will release the holder 10 (visible in FIGS. 30 through 32) as well as tension on the thread T, thus allowing the latter to be removed from the prosthesis P. However, thanks to the retention system 1000 the post members are not allowed back to the deployed position with the protrusion P1, but rather remain collapsed with the protrusion P2 by virtue of the crimping action of the thread T1000.

The view of FIG. 33, wherein the sole support structure 1 is depicted for clarity, is representative of this condition.

The prosthesis P may therefore be delivered to the implantation site and secured thereat without the need for carrying the holder 10 to the very site. Accordingly, once implantation is finalized there is no longer an issue with the holder 10 remaining at a non-accessible location in the heart of the patient. The thread T1000 may then be severed at the cutoff portion 1004, thus releasing the post members back to the protrusion P1, and removed from the prosthesis together with the retention facility 1002, which may be grasped with a surgical clamp at the rib 1006.

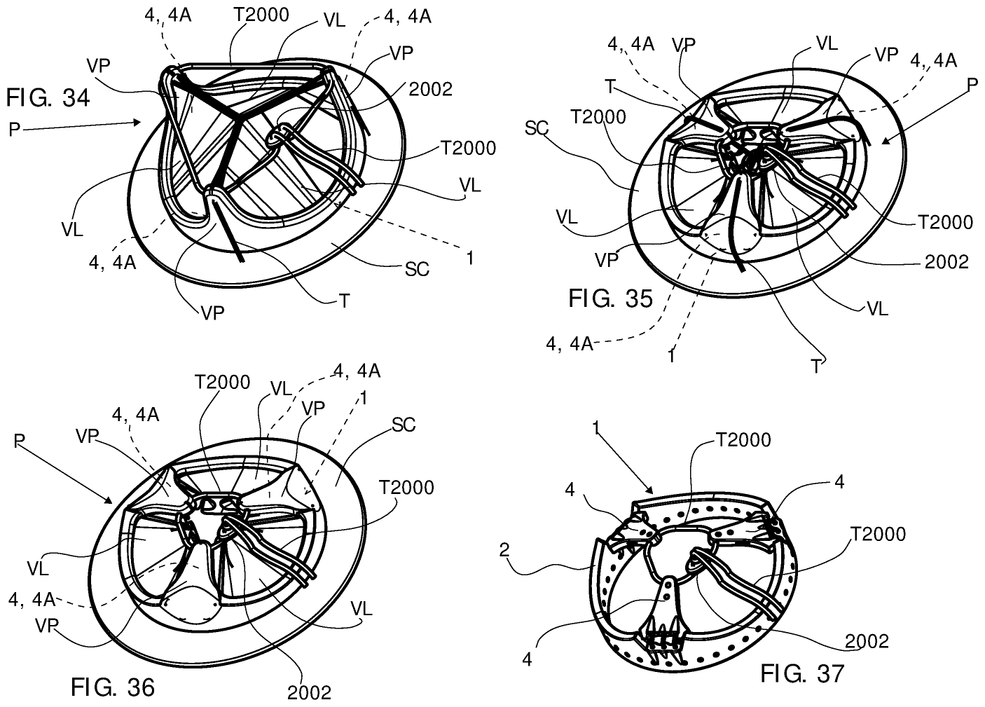

It should be noted that in embodiments—such as those of FIGS. 34 to 37—the retention facility may be provided as a simpler object than the facility 1002.

In this regard, reference number 2000 designates as a whole a second post member retention system configured to operate with the prosthesis P. The post retention system 2000 includes a retention facility 2002, and a retention thread T2000—the function of which is identical to the thread T1000. The retention facility 2002 is embodied as a pledget only featuring one or—preferably—a pair of through holes to serve as a tie-up location for the thread T2000. Operation of the retention system 2000 is identical to that of the retention system 1000, only the severing of the thread T2000 is done without the positioning aid provided by the cutoff portion 1004.

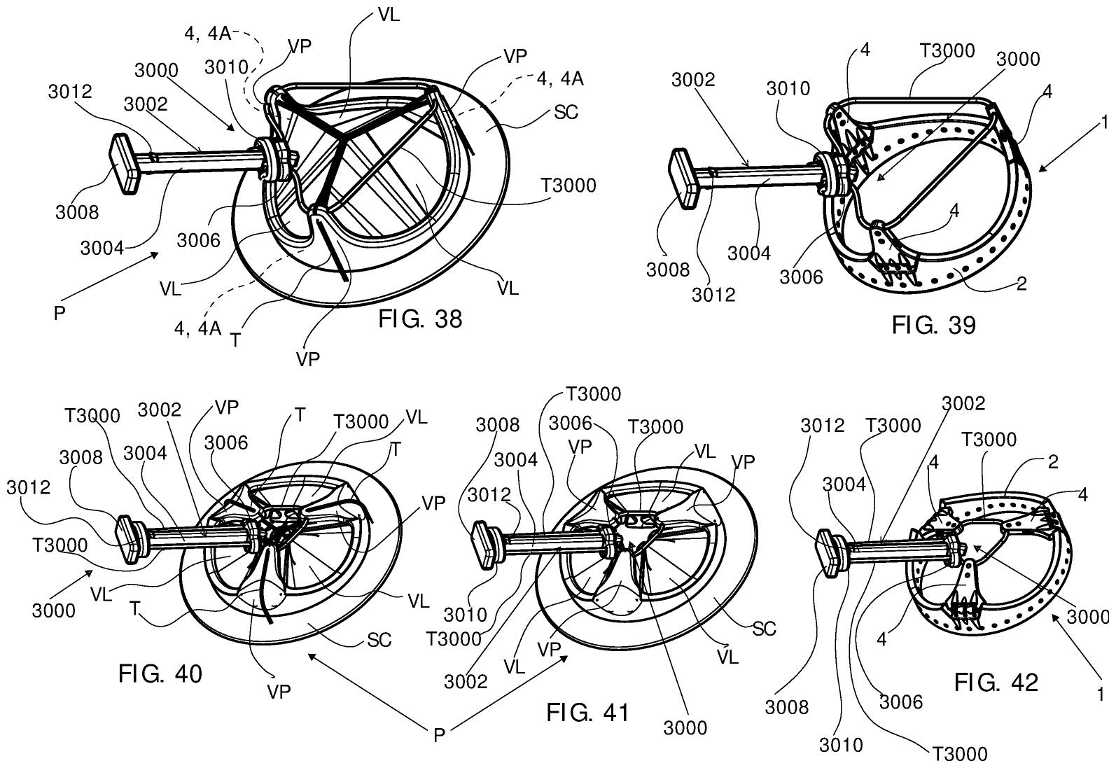

In other embodiments, such as those depicted in FIGS. 38 to 42, it is exemplified how the retention facility may deviate from a plate or plate-like design that characterizes the systems 1000 and 2000.

Reference number 3000 in such figures designates as a whole a third post member retention system configured to operate with the prosthesis P. The post retention system 3000 includes a retention facility 3002, and a retention thread T3000—the function of which is identical to the thread T1000. Unlike the systems 1000 and 2000, the retention facility 3002 includes a guide member and a mobile member (to which the retention thread T3000 is secured) displaceable over the guide member between a resting position and a tensioning position.

In detail, the retention facility 3002 includes a shaft 3004 as the guide member, the shaft 3004 comprising a first end shoulder 3006 and a second end shoulder 3008 located at opposite ends of the shaft 3004. A pledget 3010 is slidably mounted on the shaft 3004 and is movable between the shoulder 3006 (resting position) and the shoulder 3008 (tensioning position). The pledget 3010 receives free ends of the thread T3000, which is prior routed through the shoulder 3006 (which works as a loop) along two separate paths on opposite sides of the shaft 3004. A displacement of the pledget 3010 towards the shoulder 3008 thus sets tension to the thread T3000, in that the distance between the pledget 3010 and the shoulder 3006 is increased.

A bulge 3012 is formed on the shaft 3004 in proximity of the end shoulder 3008, and in particular at an axial distance along the shaft 3004 sufficient to accommodate the pledget 3010 between the bulge 3012 itself and the end shoulder 3008. In other terms, the bulge 3012 functions as a snap position lock for the pledget 3010: once moved from the shoulder 3006 to the shoulder 3008, the pledget 3010 is prevented from moving back to the shoulder 3006, and accordingly is prevented from releasing the tension on the thread T3000. The bulge 3012 is sized and dimensioned to prevent an overstepping by the pledget 3010 in the direction of the shoulder 3006 under the (design) tension of the thread T3000 when the pledget 3010 is in the tensioning position.

Operation of the system 3000 is as follows. With the post members 4, 4A collapsed to the protrusion P2 the pledget 3010 is moved to the tensioning position. The shape of the system 3000 as a whole facilitates this operation that can be performed in a syringe-like fashion, e.g. with the thumb on the shoulder 3008 and the index and middle finger astride of the pledget 3010. It should be noted, however, that the prosthesis P may be provided in a storage kit with the post members 4, 4A already collapsed to the protrusion P2 and with the retention system 3000 (as well as 1000, 2000) pre-mounted on the prosthesis with the thread T3000 (as well as T1000, T2000) in the tensioned state, whereby the practitioner has no need to operate the facility 3002.

Once the thread T is severed and the holder 10 taken away, the prosthesis P can be secured on site and the thread T3000 severed by cutting the same on either side of the shaft 3004, thereby releasing the post members 4, 4A to the protrusion P1.

By way of summary, all of the embodiments disclosed herein may be regarded as providing the following technical advantages:
i) no risk of suture entrapment during implantation owing to the variable positioning of the post members 4, 104, 204, 304 relative to the respective annular portions; in other words, each supporting structure has a variable geometry allowing to take the valve posts VP away from critical areas at the implantation site
ii) the benefit of a smaller/narrower surgical access, which is less invasive and less traumatic to the patient
iii) easy and convenient storage possibly in a blister container when storage is effected with the post members in the collapsed condition, possibly combined with dry storage and/or vacuum storage depending on the material of the valve V.

Additionally, the embodiments disclosed herein may also facilitate implantation procedures involving the use of a single loop wire snare to pull suture ends through a mechanical knot, as disclosed for instance in U.S. Pat. Nos. 5,520,702, 5,643,289, 5,669,917, 6,368,334, 6,641,592, 7,235,086 and currently commercially available through the Titanium Knot® Quick Load® unit and the Cor-Knot™ Quick Load® unit from LSI SOLUTIONS®, Victor, NY. A common difficulty with these procedures is the crimping of the fasteners at positions corresponding to the valve post members, which may at least partly hinder the routing of the unit tip to the suturing location (e.g. the sewing cuff). In the embodiments herein, transitioning the post members from the first protrusion P1 to the second protrusion P2 clears the operational area from any hinder potentially caused by the post members, while also providing an unobstructed, all-around view of the implantation site.

Naturally, while the principle of the invention remains the same, the details of construction and the embodiments may widely vary with respect to what has been described and illustrated purely by way of example, without departing from the scope of the present invention.

1. A supporting structure (1; 1A, 1B) for a prosthetic heart valve (V), the supporting structure (1; 1A, 1B) including:
an annular portion (2) defining a supporting structure longitudinal axis (XI),
a plurality of post members (4; 4A) arranged along the annular portion (2) around the supporting structure longitudinal axis (X1), each post member (4) extending from a proximal end (4P) to a distal end (4D), the distal end (4D) having a protrusion (P1, P2) with respect to the annular portion (2) and in a direction parallel to the supporting structure longitudinal axis (X1), each of the post members (4; 4A) being movable with respect to the annular portion (2; 2A) so as to vary the protrusion of the distal end (4D) thereof from a first protrusion (P1) to a second protrusion (P2), lower than the first protrusion (P1).

2. The supporting structure (1) of claim 1, further including a drive member (6) operatively connected to the post members (4) and operable to move the post members (4) between the first protrusion (P1) and the second protrusion (P2).

3. The supporting structure (1) of claim 2, further comprising a holder (10) releasably coupled to the annular portion (2).

4. (canceled)

5. The supporting structure (1; 1A; 1B) of claim 1, wherein each post member (4) is cantilevered (8) to the annular portion (2) at the proximal end (4P) thereof.

6-7. (canceled)

8. The supporting structure (1; 1A, 1B) of claim 5, wherein each of the post members (4D) is integrally formed with one or more extensions (8A) fitted into the annular portion (2; 2A, 2B), the extensions (8A) providing the cantilevered connection.

9-10. (canceled)

11. The supporting structure (1A; 1B) of claim 1, wherein the annular portion (2A; 2B) includes a distal flange (DF) at a distal end thereof (2), the distal flange including a pattern of distal sewing holes (SH_D) extending through a thickness of the distal flange (DF).

12. The supporting structure (1A) of claim 11, wherein the annular portion (2A; 2B) includes a pattern of proximal sewing holes (SH_P) at a proximal end thereof, the proximal sewing holes (SH_P) being configured for the routing of suture threads therethrough to secure a wrapping cloth or sheath around the proximal end of the annular portion.

13-19. (canceled)

20. The supporting structure (1) of claim 3, wherein the holder (10) extends across the annular portion, and the drive member (6) is attached to the holder (10) at the supporting structure longitudinal axis (X1), the drive member (6) being connected to each support post (4) by means of a respective thread (T), and the drive member (6) being configured to attract the threads (T) towards the supporting structure longitudinal axis (X1) to orient the distal ends thereof (4D) towards the supporting structure longitudinal axis (X1),
wherein each thread (T) has a first end coupled to the drive member (6), and is coupled to the respective post member (4) at the distal end (4D) thereof, and
wherein each thread (T) is coupled to the holder (10) at a second end thereof.

21-23. (canceled)

24. The supporting structure (1) of claim 20, wherein the drive member comprises a rotary ratchet drive member,
wherein the first end of each thread (T) is coupled to the rotary ratchet drive member, and
wherein the rotary ratchet drive member has a drive hub (16) configured to be engaged by a drive instrument for operation of the drive member (6).

25-26. (canceled)

27. The supporting structure (1) of claim 20, wherein the second ends of each thread are loop-tied to the holder (10A, 10B, 10C) to expose a cutoff section, wherein cutting of each thread (T) at the cutoff section releases the coupling between the holder (10) and the supporting structure (1), and
wherein cutting of each thread (T) at the cutoff section also leaves the post members (4) in a position having the first protrusion (P1).

28-41. (canceled)

42. A heart valve prosthesis (P), comprising a prosthetic heart valve (V) coupled to the supporting structure (1) of claim 3.

43. The heart valve prosthesis (P) of claim 42, wherein the prosthetic heart valve (V) including a valve annulus (VA) defining a valve longitudinal axis (XV) and a plurality of prosthetic valve leaflets (VL) extending between subsequent valve posts (VP) arranged along the prosthetic annulus (VA) and around the valve axis (VX),
wherein the valve posts (VP) are fixed to the post members (4) of the supporting structure, and
wherein the valve annulus (VA) is fixed to the annular portion (2) of the supporting structure (1).

44-46. (canceled)

47. The heart valve prosthesis (P) of claim 42, wherein the prosthetic heart valve (V) comprises a biological tissue.

48. (canceled)

49. The heart valve prosthesis of claim 43, wherein the prosthetic valve leaflets (VL) comprise a decellularized sterilized mammalian tissue having an extracellular matrix, wherein a plurality of interstitial spaces of the extracellular matrix include a solution of one or more polyols.

50. (canceled)

51. A kit including:
the heart valve prosthesis of claim 42 with the post members (4) of the supporting structure (1) having the second protrusion (P2),
the supporting structure,
a retention system (1000; 2000; 3000) coupled to the prosthesis (P), the retention system including a retention facility (1002; 2002; 3002), and a retention thread (T1000; T2000; T3000), the retention thread (T1000; T2000; T3000) configured to be routed through the distal ends (4D) of the post members (4; 4A) and secured to the retention facility in a tensioned state to prevent the post members (4; 4A) to transition to the first protrusion (P1).

52-61. (canceled)
